(19) United States
(12) Patent Application Publication
(10) Pub. No.: US 2025/0073223 A1
(43) Pub. Date: Mar. 6, 2025

(54) TARGETED NANOCARRIERS FOR SYSTEMIC DELIVERY OF IRAK4 INHIBITORS TO INFLAMED TISSUES

(71) Applicants: Oregon State University, Corvallis, OR (US); Oregon Health & Science University, Portland, OR (US)

(72) Inventors: Oleh Taratula, Corvallis, OR (US); Youngrong Park, Corvallis, OR (US); Olena Taratula, Corvallis, OR (US); Tetiana Korzun, Portland, OR (US); Daniel L. Marks, Portland, OR (US)

(73) Assignees: Oregon State University, Corvallis, OR (US); Oregon Health & Science University, Portland, OR (US)

(21) Appl. No.: 18/823,280

(22) Filed: Sep. 3, 2024

Related U.S. Application Data
(60) Provisional application No. 63/580,122, filed on Sep. 1, 2023.

(51) Int. Cl. A61K 31/4725; A61K 9/51; A61P 1/00; A61P 29/00

(52) U.S. Cl. CPC A61K 31/4725; A61K 9/5153; A61P 1/00; A61P 29/00

(57) ABSTRACT

A nanoparticle for delivering an interleukin-1 receptor-associated kinase 4 (IRAK4) inhibitor to a subject and methods for treating inflamed tissue in a subject using the nanoparticle.

### CROSS-REFERENCE TO RELATED APPLICATION

This application claims the benefit of U.S. Patent Application No. 63/580,122, filed Sep. 1, 2023, expressly incorporated herein by reference in its entirety.

### STATEMENT OF GOVERNMENT LICENSE RIGHTS

This invention was made with Government support under Contract Nos. R01CA237569, R01HD101450, R37CA234006, and TL1TR002371 awarded by National Institutes of Health. The Government has certain rights in the invention.

### STATEMENT REGARDING SEQUENCE LISTING

The Sequence Listing XML associated with this application is provided in XML format and is hereby incorporated by reference into the specification. The name of the XML file containing the sequence listing is 3014-P32US_Seq_List_20240903. The XML file is 1,970 bytes; was created on Sep. 3, 2024; and is being submitted electronically via Patent Center with the filing of the specification.

### BACKGROUND

The inflammatory response is the natural protective mechanism of the body against injury and infection. In certain instances, however, persistent and uncontrolled inflammation becomes detrimental and contributes to the development and progression of different debilitating diseases, such as atherosclerosis, cancer, autoimmune and neurodegenerative disorders. Anti-inflammatory drugs are considered a viable strategy for preventing and treating inflammation. However, many of these small-molecule drugs exhibit limited aqueous solubility in physiologic fluids, in vivo instability, and non-specific biodistribution, resulting in relatively low bioavailability, off-target effects, and modest therapeutic efficacy. Although currently available nanoparticle-based drug delivery systems can address some of these limitations, their efficient systemic distribution to inflamed tissue remains challenging. To mitigate this issue, some nanoparticles have been designed to deliver anti-inflammatory drugs via local routes, including oral administration for inflammatory bowel disease, intra-articular injection for arthritis, and intranasal administration for lung inflammation. However, these delivery strategies are limited to the treatment of accessible inflammation sites. Therefore, nanocarriers that efficiently accumulate in inflamed tissues following systemic administration are essential.

The most frequently used classes of medication to treat inflammation are steroid-based and non-steroidal anti-inflammatory drugs (NSAIDs). However, they are associated with a number of adverse effects, including weight gain, high blood pressure, and hyperglycemia in the case of steroids and gastrointestinal and cardiovascular problems in the case of NSAIDs. New anti-inflammatory therapeutic agents are also being developed to inhibit enzymes, typically kinases, including inhibitors of interleukin-1 receptor-associated kinase 4 (IRAK4). IRAK4 is a critical signal transducer downstream of interleukin-1 receptor (IL-1R), IL-18 receptor (IL-18R), and toll-like receptors (TLRs) in innate inflammation signaling. The recruitment of IRAK4 occurs after MyD88 (myeloid differentiation primary response protein 88) binds to TLRs and other receptors. TLRs are involved in a variety of disease processes, including infection response and various auto-inflammatory disorders. IRAK4 activates downstream molecules involved in cytokine and inflammatory responses, such as JNK, NF-KB, and p38 MAPK. As a result, controlling IRAK4 activity is a promising therapeutic strategy for a variety of inflammatory diseases. Moreover, despite the fact that IRAK4 is involved in a variety of signaling inflammatory pathways, adults with IRAK4 deficiency are not susceptible to infections caused by fungi, parasites, or bacteria because other innate immune pathways serve as the first line of defense. Therefore, IRAK4 inhibition can potentially provide the suppression of inflammatory responses while maintaining adequate levels of protection against microbial infections. Localized and efficient inhibition of IRAK4 activity is an attractive therapeutic target because the signaling pathways downstream of TLR activation are suppressed and the production of inflammatory cytokines such as tumor necrosis factor (TNF-α), interleukin (IL)-6, and interleukin (IL)-1β is greatly attenuated. These cytokines play a significant role in propagating and amplifying inflammatory signals in inflamed tissues.

Despite the advances in the development of IRAK4 inhibitors that suppress inflammatory responses while maintaining adequate levels of protection against microbial infections, a need exists to develop improved drug carriers for efficient and localized delivery of available IRAK4 inhibitors to inflamed tissues. The present disclosure fulfills this need and provides further related advantages.

### SUMMARY

In one aspect, the disclosure provides nanoparticle for delivering an interleukin-1 receptor-associated kinase 4 (IRAK4) inhibitor to inflamed tissue. In certain embodiment, the nanoparticle comprises:
(a) a first amphiphilic block polyethylene glycol-polycaprolactone (PEG-PCL) copolymer having a hydrophilic PEG block and a hydrophobic PCL block, and
(b) a second amphiphilic block polyethylene glycol-polycaprolactone (PEG-PCL) copolymer having a hydrophilic PEG block and a hydrophobic PCL block, and an IRAK4 targeting moiety covalently coupled to the terminus of the hydrophilic PEG block,
wherein the first and second copolymers form a nanoparticle having a bilayer structure with an inner hydrophobic layer (or core) formed from the first and second hydrophobic PCL blocks and an outer hydrophilic layer (or shell) formed by the first and second hydrophilic PEG blocks; and
(c) an IRAK4 inhibitor encapsulated in the hydrophobic layer (or core).

In certain embodiments, the ratio of the first amphiphilic block polyethylene glycol-polycaprolactone (PEG-PCL) copolymer to the second amphiphilic block polyethylene glycol-polycaprolactone (PEG-PCL) copolymer is about 9:1 (weight:weight).

In certain embodiments, the IRAK4 inhibitor is zimlovisertib (PF-06650833).

In certain embodiments, the targeting moiety is a peptide having a binding affinity to a vascular cell adhesion molecule 1 (VCAM1). In certain of these embodiments, the targeting moiety is VHPKQHRGGSKGC (SEQ ID NO: 1).

In a related aspect, the disclosure provides a pharmaceutical composition comprising the nanoparticle described herein and a pharmaceutically acceptable carrier.

In other aspects, methods for using the nanoparticle described herein are provided. In certain embodiments, the disclosure provides methods for delivery of an interleukin-1 receptor-associated kinase 4 (IRAK4) inhibitor to inflamed tissue in a subject. In other embodiments, the disclosure provides methods for treating inflamed tissue in a subject. In further embodiments, the disclosure provides methods for treating colitis in a subject. In the methods, a therapeutically effective amount of the nanoparticle described herein is administered to a subject (e.g., human) in need thereof. In certain of these embodiments, administering the nanoparticle comprises systemic administration.

### DESCRIPTION OF THE DRAWINGS

The foregoing aspects and many of the attendant advantages of this invention will become more readily appreciated as the same become better understood by reference to the following detailed description, when taken in conjunction with the accompanying drawings.

FIGS. 1A and 1B show a schematic illustration of a two-step preparation procedure for a representative nanocarrier of the disclosure: VCAM1-targeted nanocarriers loaded with the IRAK4 inhibitor (VCAM1-IRAK4 NC). FIGS. 1A shows the coupling of the VCAM1 peptide to maleimide-PEG-PCL via a thioether bond. FIG. 1B shows the loading of the IRAK4 inhibitor in polymeric nanocarriers composed of the VCAM1-PEG-PCL and CH₃O-PEG-PCL.

FIGS. 2A-2C compare cryo-TEM images and DLS profiles of non-targeted IRAK4 NC (FIG. 2A), a representative nanocarrier of the disclosure, VCAM1-targeted IRAK4 NC (FIG. 2B) and DLS profiles of IRAK4 NC and VCAM1-IRAK4 NC (FIG. 2C).

FIGS. 3A-3H illustrate cellular uptake properties of a representative nanocarrier of the disclosure. Vcam1 mRNA levels in CCD-18Co cells treated with PBS or LPS (FIG. 3A). Flow cytometry analysis of cellular internalization efficiency of non-targeted and VCAM1-targeted nanocarriers by LPS treated (+) and non-treated (−) CCD-18Co cells after 24 hours of incubation FIG. 3B). Black bar is the fluorescence of cells without NCs (negative control). Flow cytometry histograms of CCD-18Co cells preincubated with PBS (FIG. 3C) or LPS (FIG. 3D) and treated with non-targeted nanocarriers and VCAM1-targeted nanocarriers for 24 hours. Black curve represents negative control (FIGS. 3C and 3D). Representative fluorescence microscopy images of the PBS (no treatment) or LPS-treated cells that were incubated with either non-targeted or VCAM1-targeted nanoparticles for 24 hours (FIGS. 3E-3H). Scale bar=200 μm. The results are represented as the mean±standard deviation. Statistical comparisons between groups were executed with one-way ANOVA (*p<0.05, **p<0.01, ***p<0.001).

FIG. 4A compares percent change of body weights of control mice and mice treated intravenously with a representative nanocarrier of the disclosure, VCAM1-IRAK4 NC, (23 mg kg⁻¹) 4 times every other day. The values are represented as the mean±standard deviation. Statistical comparisons between groups were performed with repeated measures unpaired Student's t-test (n=4). FIGS. 4B-4D compare serum concentrations of biochemical markers (ALP, ALT, CK, BUN, Cr), proteins, and electrolytes in control and VCAM1-IRAK4 NC treated mice. Serum samples were collected on Day 10. The values are presented as the mean±standard deviation. Statistical comparisons between groups were performed with unpaired Student's t-test.

FIG. 5A are representative photographs of the right paws taken before and after the injection of λ-carrageenan. Percent change of paw thickness of mice treated with saline, IRAK4 NC (11.5 mg kg⁻¹), and a representative nanocarrier of the disclosure, VCAM1-IRAK4 NC (11.5 mg kg⁻¹), (n=3) (FIG. 5B). Representative fluorescence images of the shaved mice receiving systemic administration of SiNc-loaded IRAK4 NC and VCAM1-IRAK4 NC 1 hour before λ-carrageenan administration (FIG. 5C). Images were taken 2 and 3 hours after administration of nanocarriers. Mean fluorescence intensities in the inflamed paws 3 hours after IRAK4 NC and VCAM1-IRAK4 NC administration (n=4) (FIG. 5D). The unpaired t-test was used for statistical comparisons (**p<0.01). Relative qRT-PCR quantification of Tnf, Il1β, and Il6 mRNA in the paws of each group (FIG. 5E). The values are presented as the mean±standard deviation. Statistical comparisons between groups were performed with one-way ANOVA (*p<0.05, **p<0.01).

FIG. 6A compare fluorescence images of the shaved mice with DSS-induced colitis 24 hours after IV injection with SiNc-loaded IRAK4 NC and a representative nanocarrier of the disclosure, VCAM1-IRAK4 NC. NIR fluorescence images of the colons resected from DSS-induced colitis mice 24 hours after IV injection with saline (DSS+Saline), SiNc-loaded IRAK4 NC (DSS+Non-targeted NC), and SiNc-loaded VCAM1-IRAK4 NC (DSS+VCAM1 NC) (n=3) (FIG. 6B). Mean fluorescence intensity of the colons from each treatment group (FIG. 6C). NIR fluorescence images of major organs resected from DSS-induced colitis mice 24 hours after IV injection with SiNc-loaded IRAK4 NC (DSS+Non-targeted NC), and SiNc-loaded VCAM1-IRAK4 NC (DSS+VCAM1 NC) (n=3) (FIG. 6D). Mean fluorescence intensity of the major organs from each treatment group (FIG. 6E). The values are presented as the mean±standard deviation. Statistical comparisons between groups were performed with one-way ANOVA (*p<0.05).

FIGS. 7A-7E present data illustrating the effectiveness of a representative nanocarrier of the disclosure, VCAM1-IRAK4 NC, for the treatment of colitis. FIG. 7A shows photographs of colons excised from healthy mice (Control), and DSS-induced colitis mice treated with saline (DSS) and VCAM1-IRAK4 NC (DSS+VCAM1-IRAK4 NC). The average colon lengths of each group (FIG. 7B). Daily body weight changes in each group for 10 days (FIG. 7C). Colon damage scores and representative H&E images of stained colon sections (FIGS. 7D and 7E). Scale bar=200 μm. The values are presented as the mean±standard deviation. Statistical comparisons between groups were performed with one-way ANOVA (n=4, *p<0.05, **p<0.01, ***p<0.001).

FIGS. 8A and 8C compare fluorescence microscope images of sectioned colon tissues that have been stained for IL-6 (FIG. 8A) and TNF-α (FIG. 8C). Scale bars=200 μm. Examined colons were collected from healthy mice (Control), and DSS-induced colitis mice treated with saline (DSS) and a representative nanocarrier of the disclosure, VCAM1-IRAK4 NC (DSS+VCAM1-IRAK4 NC). Mean fluorescence signals from the mouse colons stained for IL-6 (FIG. 8B) and TNF-α (FIG. 8D), respectively. The values are presented as the mean±standard deviation. Statistical comparisons between groups were performed with one-way ANOVA (n=4, **p<0.01, ***p<0.001).

FIGS. 9A and 9B compare IL-6 and TNF-α protein levels, respectively, in colons excised from healthy mice (Control), and DSS-induced colitis mice treated with saline (DSS) and a representative nanocarrier of the disclosure, VCAM1-IRAK4 NC (DSS+VCAM1-IRAK4 NC).

FIGS. 10A-10C illustrate properties of a representative IRAK4-loaded nanocarrier of the disclosure: the average hydrodynamic size (FIG. 10A), polydispersity index (PDI) (FIG. 10B), and zeta potential (FIG. 10C) of IRAK4-loaded nanocarriers recorded from Day 0 to Day 16 (n=3). Samples were stored at room temperature and evaluated using DLS for 16 days. The results are represented as the mean±standard deviation.

FIG. 11 illustrates the release profile of the IRAK4 inhibitor from a representative IRAK4-loaded nanocarrier of the disclosure evaluated using dialysis under sink conditions in PBS (pH=7.4) at 37° C. The results are represented as the mean±standard deviation (n=3).

FIG. 12 compares fluorescence date for treated and non-treated cells. Mean fluorescence intensity in LPS treated (+) and non-treated (−) CCD-18Co cells incubated with Nile Red-loaded non-targeted and VCAM1-targeted nanocarriers for 24 hours. The fluorescence signal in the microscopy images was quantified using the ImageJ software. The results are represented as the mean±standard deviation. Statistical comparisons between groups were executed with one-way ANOVA (***p<0.001, ****p<0.0001).

FIGS. 13A and 13B compare fluorescence images of the resected organ receiving systemic administration of SiNc-loaded IRAK4 NC and a representative nanocarrier of the disclosure, VCAM1-IRAK4 NC, 1 hour before λ-carrageenan administration (FIG. 13A). Images were taken 6 hours after administration of nanocarriers. Mean fluorescence intensities in the organs 6 hours after administration of nanocarriers (=4) (FIG. 13B). The results are represented as the mean±standard deviation.

FIGS. 14A and 14C are fluorescence microscope images of sectioned paw edema tissues that have been stained for IL-6 and TNF-α, respectively. Scale bars=50 μm. Examined paws were collected from carrageenan-induced paw edema mice treated with saline, IRAK4 NC, and VCAM1-IRAK4 NC. FIGS. 14B and 14D compare mean fluorescence signals from the paw edema tissues stained for IL-6 and TNF-α, respectively. The values are presented as the mean±standard deviation. Statistical comparisons between groups were performed with one-way ANOVA (n=3, *p<0.05, **p<0.01, ****p<0.0001).

FIG. 15A compares photographs and average lengths of colons excised from healthy mice (Control), DSS-induced colitis mice treated with saline (DSS), and free IRAK4 inhibitor (DSS+IRAK4 inhibitor). FIGS. 15B-15D are H&E images of stained colon sections from healthy mice (Control) (FIG. 15B), DSS-induced colitis mice treated with saline (DSS) (FIG. 15C), and free IRAK4 inhibitor (DSS+IRAK4 inhibitor) (FIG. 15D). Scale bar=200 μm. The values are presented as the mean±standard deviation. Statistical comparisons between groups were performed with one-way ANOVA (n=4, ***p<0.001).

### DETAILED DESCRIPTION

Persistent and uncontrolled inflammation is the root cause of various debilitating diseases. Given that interleukin-1 receptor-associated kinase 4 (IRAK4) is a critical modulator of inflammation, inhibition of its activity with selective drug molecules (IRAK4 inhibitors) represents a promising therapeutic strategy for inflammatory disorders. To exploit the full potential of this treatment approach, drug carriers for efficient delivery of IRAK4 inhibitors to inflamed tissues are essential.

The present disclosure provides a nanoparticle-based platform for the targeted systemic delivery of hydrophobic therapeutic agents to tissues.

In one aspect, the disclosure provides a nanocarrier (i.e., nanoparticle) for delivering a hydrophobic therapeutic agent to a targeted tissue. The nanoparticles described herein are polymeric nanoparticles formed from amphiphilic copolymers and include a hydrophobic therapeutic agent. By virtue of a targeting moiety associated with the nanoparticle (e.g., present on the nanoparticle surface), the nanoparticle is directed to a target where the nanoparticle's hydrophobic therapeutic agent cargo is delivered.

In certain embodiments, the nanoparticle comprises:
(a) a first amphiphilic block polyethylene glycol-polycaprolactone (PEG-PCL) copolymer having a hydrophilic PEG block and a hydrophobic PCL block, and
(b) a second amphiphilic block polyethylene glycol-polycaprolactone (PEG-PCL) copolymer having a hydrophilic PEG block and a hydrophobic PCL block, and a targeting moiety covalently coupled to the terminus of the hydrophilic PEG block,
wherein the first and second copolymers assemble into (i.e., form) a nanoparticle having a bilayer structure with an inner hydrophobic layer (or core) formed from the first and second hydrophobic PCL blocks and an outer hydrophilic layer (or shell) formed by the first and second hydrophilic PEG blocks; and
(c) a hydrophobic therapeutic agent encapsulated in the hydrophobic layer or core).

The hydrophobicity of a compound can be characterized by the partition coefficient (P=[organic]/[aqueous]), which is a measure of lipophilicity and is defined as the ability of a compound to differentially dissolve in a mixture of water and lipids/organic solvents.

The log10 value of P (logP) is a constant and is a negative value for hydrophilic compounds (higher affinity for the aqueous phase), a positive value for lipophilic compounds (higher affinity for the lipid/organic solvent phase), and zero (0) value for compounds which partition equally between lipid and aqueous phases. For example, logP=1 means that the partitioning of the compound is 10 times more in lipid/organic solvent phase compared to aqueous phase.

As used herein, the term “hydrophobic therapeutic agent” refers to a therapeutic agent having a positive logP value (i.e., logP>0), which means that the compound prefers to be dissolved in oil phase. The higher logP value, the more hydrophobic the compound is. Theoretically, the maximum logP value can be up to 7. Extremely water-insoluble compounds have logP values between 3 and 4.

IRAK4 inhibitor zimlovisertib (PF-06650833) has a computationally predicted logP values of 1.05 (from Chemaxon) and 1.75 (from ALOGPS).

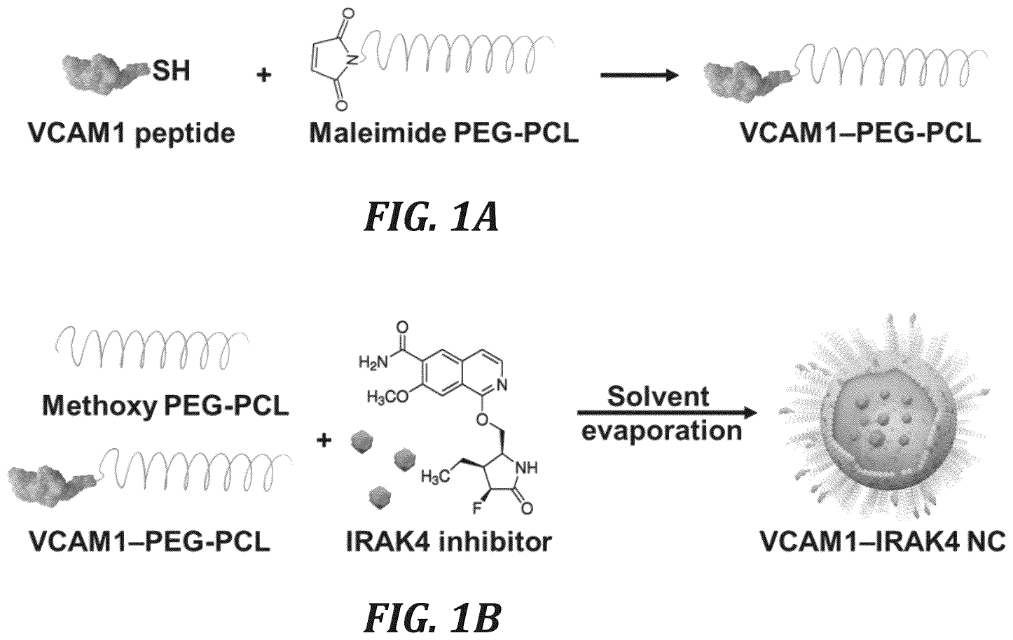

As noted above, the nanoparticles described herein are used for the targeted systemic delivery of hydrophobic therapeutic agents to tissues by virtue of the nanoparticle's targeting moiety. The targeting moiety is present on the nanoparticle surface as a result of assembly of the amphiphilic copolymers making up the nanoparticle. The targeting moiety is covalently coupled to the terminus of the of the hydrophilic PEG block of the second amphiphilic block polyethylene glycol-polycaprolactone (PEG-PCL) copolymer. FIG. 1A is a schematic illustration of the coupling of a representative targeting moiety (VCAM1) to the terminus of the PEG block of a PEG-PCL copolymer. FIG. 1B is a schematic illustration of the formation (e.g., assembly) of a nanoparticle of the disclosure with the incorporation of a representative hydrophobic therapeutic agent (e.g., IRAK4 inhibitor: zimlovisertib (PF-06650833)). As shown in FIG. 1B, the representative targeting moiety is present on the nanoparticle surface.

The targeting moiety serves to selectively direct the nanoparticle with its therapeutic agent cargo to a tissue to be treated. The targeting moiety is a moiety having an affinity to the tissue to be treated. In certain embodiments, the targeting moiety may be a ligand for a cell receptor expressed by the tissue to be treated. Suitable targeting moieties and their targets are known in the art.

In other aspects, the present disclosure provides methods for delivery of a hydrophobic therapeutic agent to a select tissue in a subject using the nanoparticle described herein. In related aspects, by virtue of the targeted delivery of the therapeutic agent cargo of the nanoparticle to a tissue to be treated, the present disclosure also provides methods for treating conditions and diseases treatable by administration of the therapeutic agent carried by the nanoparticle.

In one embodiment, the present disclosure provides a nanoparticle for delivering an interleukin-1 receptor-associated kinase 4 (IRAK4) inhibitor to inflamed tissue. In certain embodiments, the nanoparticle comprises:
(a) a first amphiphilic block polyethylene glycol-polycaprolactone (PEG-PCL) copolymer having a hydrophilic PEG block and a hydrophobic PCL block, and
(b) a second amphiphilic block polyethylene glycol-polycaprolactone (PEG-PCL) copolymer having a hydrophilic PEG block and a hydrophobic PCL block, and an IRAK4 targeting moiety covalently coupled to the terminus of the hydrophilic PEG block,
wherein the first and second copolymers assemble into/form a nanoparticle having a bilayer structure with an inner hydrophobic layer (or core) formed from the first and second hydrophobic PCL blocks and an outer hydrophilic layer (or shell) formed by the first and second hydrophilic PEG blocks; and
(c) an IRAK4 inhibitor encapsulated in the hydrophobic layer (or core).

In certain of these embodiments, the ratio of the first amphiphilic block polyethylene glycol-polycaprolactone (PEG-PCL) copolymer to the second amphiphilic block polyethylene glycol-polycaprolactone (PEG-PCL) copolymer is from about 9:1 (weight:weight).

In certain embodiments, the hydrophilic PEG block of the first and second copolymers has a molecule weight from about 2 kDa to about 10 kDa. In certain of these embodiments, the hydrophilic PEG block has a molecule weight of about 2 kDa.

In certain embodiments, the hydrophobic PCL block of the first and second copolymers has a molecule weight from about 5 kDa to about 15 kDa. In certain of these embodiments, the hydrophobic PCL block has a molecule weight of about 5 kDa.

In one embodiment, the hydrophilic PEG block of the first and second copolymers has a molecule weight of about 2 kDa and the hydrophobic PCL block has a molecule weight of about 5 kDa.

In certain embodiments, the therapeutic agent-containing nanoparticle contains from about 3 to about 5 mg therapeutic agent per 50 mg nanoparticle. In other embodiments, therapeutic agent-containing nanoparticle contains from about 1 to about 5 mg therapeutic agent.

In certain embodiments, the nanoparticle described herein has a hydrodynamic size from about 10 to about 200 nm. In other embodiments, the nanoparticle described herein has a hydrodynamic size from about 30 to about 100 nm.

In certain embodiments, the nanoparticle described herein has a polydispersity index from about 0.001 to about 0.300. In other embodiments, the nanoparticle described herein has a polydispersity index from about 0.001 to about 0.200.

In certain embodiments, the IRAK4 inhibitor is zimlovisertib (PF-06650833).

In certain embodiments, the targeting moiety is a peptide having a binding affinity to a vascular cell adhesion molecule 1 (VCAM1). In certain of these embodiments, the targeting moiety is VHPKQHRGGSKGC (SEQ ID NO: 1).

In another aspect, the present disclosure provides a pharmaceutical composition comprising the nanoparticle described herein and a pharmaceutically acceptable carrier or diluent. Suitable carriers include carriers suitable for systemic administration, such as intravenous or subcutaneous injection. Representative pharmaceutical compositions include solutions for injection, such as saline and dextrose solutions.

In further aspects, methods for using the nanoparticle described herein are provided.

In certain embodiments, the present disclosure provides a method for delivery of an interleukin-1 receptor-associated kinase 4 (IRAK4) inhibitor to inflamed tissue. In these embodiments, a therapeutically effective amount of the nanoparticle described herein (i.e., comprising an IRAK4 inhibitor) is administered to a subject in need thereof.

In other embodiments, the present disclosure provides a method for treating inflamed tissue in a subject. In these embodiments, a therapeutically effective amount of the nanoparticle described herein (i.e., comprising an IRAK4 inhibitor) is administered to a subject in need thereof.

In other embodiments, the present disclosure provides a method for treating colitis in a subject. In these embodiments, a therapeutically effective amount of the nanoparticle described herein (i.e., comprising an IRAK4 inhibitor) is administered to a subject in need thereof.

In the methods described herein, the therapeutic agent-containing nanoparticle is systemically administered by, for example, intravenous or subcutaneous injection.

In certain of the methods described herein, the subject is a human.

The following describes the preparation, characterization, and effectiveness of a representative nanoparticle-based carrier (i.e., nanocarrier) for the targeted systemic delivery of an IRAK4 inhibitor.

In one embodiment, the disclosure provides a nanoparticle-based carrier for the targeted systemic delivery of clinically tested IRAK4 inhibitor, zimlovisertib (PF-06650833), which has limited aqueous solubility (57 μg mL⁻¹). This nanocarrier increases the intrinsic aqueous dispersibility of this IRAK4 inhibitor by 40 times. A targeting peptide on the surface of nanocarrier significantly enhances their accumulation after intravenous injection in inflamed tissues of mice with induced paw edema and ulcerative colitis when compared to non-targeted counterparts. The delivered IRAK4 inhibitor markedly abates inflammation and dramatically suppresses paw edema, mitigates colitis symptoms, and reduces proinflammatory cytokine levels in the affected tissues. Importantly, repeated injections of IRAK4 inhibitor-loaded nanocarriers have no acute toxic effect on major organs of mice. Therefore, the nanocarriers described herein have the potential to significantly improve the therapeutic efficacy of IRAK4 inhibitors for different inflammatory diseases.

The nanoparticle-based carrier described herein provides effective targeted delivery of an IRAK4 inhibitor (e.g., zimlovisertib (PF-06650833)) to inflamed tissues after systemic administration. PF-06650833 is a small molecule with excellent IRAK4 selectivity and the lowest IC₅₀ value of 0.2 nM reported to date. PF-06650833 is the first IRAK4 inhibitor to undergo human clinical trials for safety profiles and rheumatoid arthritis treatment after oral administration. In two phase I clinical trials, PF-06650833 was well tolerated and no dose-limiting side effects were observed. However, its limited aqueous solubility (57 μg mL⁻¹) resulted in low absolute oral bioavailability, and the potential therapeutic effects of systemic IRAK4 inhibition remain unclear. The nanoparticle-based carrier described herein increases the aqueous dispersibility of the encapsulated PF-06650833 by 40 times and provided efficient and targeted delivery to inflamed tissues of mice with induced paw edema and in dextran sulfate (DSS)-induced ulcerative colitis following intravenous injection. The delivered IRAK4 inhibitor exhibited a significant anti-inflammatory effect without obvious systemic toxicity.

### Preparation of VCAM1-Targeted Nanocarriers for Systemic Delivery of IRAK4 Inhibitor

To improve its aqueous dispersibility, circulation time in the blood, and passive accumulation in inflamed tissue, the IRAK 4 inhibitor, PF-06650833 (FIG. 1B), was encapsulated in the hydrophobic core of a poly (ethylene glycol)-block-poly(-caprolactone) (PEG-PCL)-based nanocarrier using a solvent evaporation approach. Numerous preclinical studies demonstrated that PEG-PCL-based nanoparticles hold great potential for drug delivery due to their safety, biocompatibility, and ability to effectively transport hydrophobic payloads following systemic administration. PEG and PCL are biocompatible and nontoxic materials approved for biomedical uses in humans by the US Food and Drug Administration. PEG-PCL copolymers are biodegradable, easy to synthesize, and form nanoparticles with hydrophobic cores that can be used to encapsulate a variety of compounds with limited aqueous solubility. The versatility of these copolymers, encompassing a variety of functional groups, facilitates their chemical conjugation to different targeting ligands, thus allowing the production of nanoparticles targeted to various cellular receptors. PEG-PCL nanoparticles exhibit prolonged systemic circulation and effectively deliver therapeutic and imaging agents to diseased tissues with leaky vasculature through passive targeting. According to previous research, angiogenesis also contributes to the development of inflammatory disorders (e.g., inflammatory bowel disease) and growth factors such as VEGF cause blood vessel leakiness in inflamed tissues. To increase the specificity and internalization efficiency of our developed nanocarriers into inflamed cells, their surface was functionalized with a peptide (VHPKQHRGGSKGC (SEQ ID NO: 1)) exhibiting high binding affinity to a vascular cell adhesion molecule 1 (VCAM1). VCAM1 is one of the endothelial cell adhesion molecules that is overexpressed in inflamed endothelial cells and serves to recruit leukocytes to the inflamed region. VCAM1 is also well-suited for targeting nanoparticles to inflammation sites due to its low basal expression in various organs that are not inflamed.

FIGS. 1A and 1B show a schematic illustration of a two-step preparation procedure for a representative nanocarrier of the disclosure: VCAM1-targeted nanocarriers loaded with the IRAK4 inhibitor (VCAM1-IRAK4 NC). In the first step, maleimide-PEG-PCL was functionalized with the VCAM1 peptide by conjugating the maleimide group of the polymer to the sulfhydryl group of the cysteine in the peptide sequence (FIG. 1A). The conjugation yield of this reaction was 96.5%, as determined by the Measure-it™ thiol quantification kit. Afterward, a mixture of the prepared VCAM1-PEG-PCL and methoxy PEG-PCL (CH₃O-PEG-PCL) was used at a 1:9 ratio to formulate VCAM1-targeted nanocarriers loaded with the IRAK4 inhibitor, PF-06650833 (VCAM1-IRAK4 NC, FIG. 1B). Additionally, non-targeted nanocarriers (IRAK4 NC) were formulated using only CH₃O-PEG-PCL without VCAM1 conjugated to PEG-PCL (VCAM1-PEG-PCL). In one embodiment, the nanoparticles described having a drug loading efficiency of 76.5% were able to disperse 2.3 mg of PF-06650833 in 1 mL of saline solution and thereby increasing its aqueous dispersibility (57 μg mL⁻¹) by 40 times.

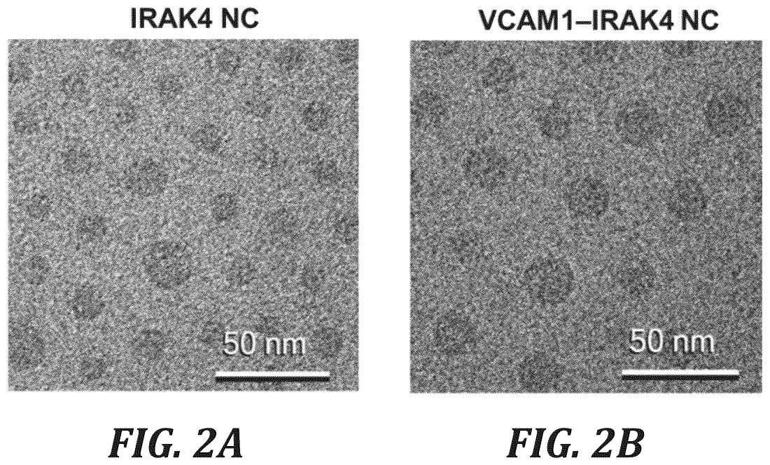

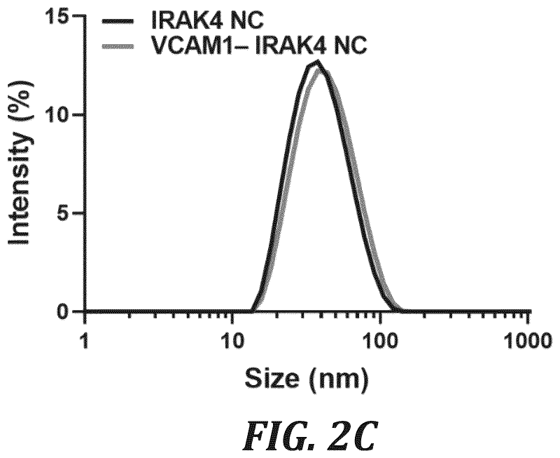

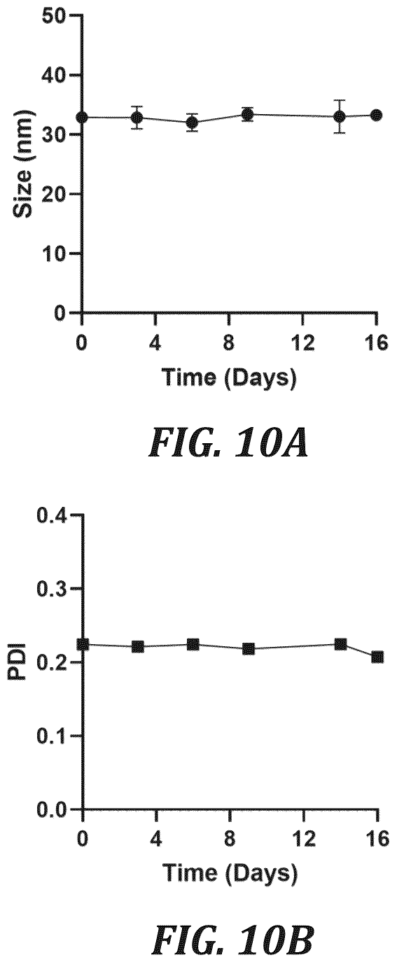

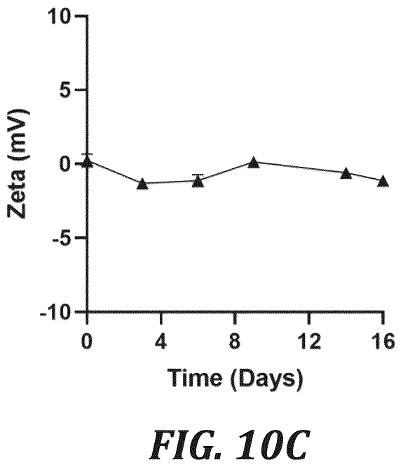

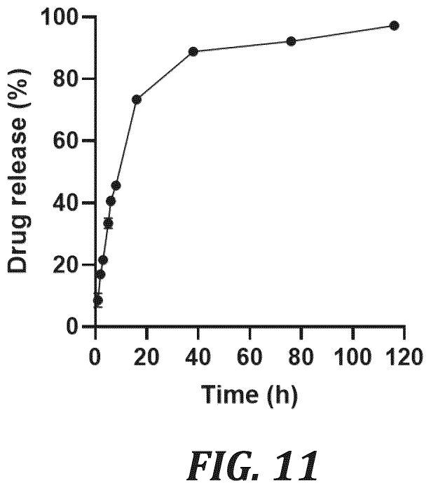

Cryo-TEM images indicate that both VCAM1-IRAK4 NC and non-targeted IRAK4 NC have a spherical shape (FIGS. 2A and 2B). The average hydrodynamic diameter and polydispersity index (PDI) of VCAM1-IRAK4 NC (39.9±0.4 nm, PDI=0.21) were comparable to non-targeted IRAK4 NC (36.5±0.7 nm, PDI=0.18), as determined by dynamic light scattering (DLS) (FIGS. 2C). Finally, zeta potential measurements revealed that both VCAM1-targeted (−3.85±0.59 mV) and non-targeted nanocarriers (−5.89±3.99) have a slightly negative surface charge. The stability studies revealed that no significant changes in hydrodynamic diameter, PDI, or zeta potential of IRAK4-loaded nanocarriers occurred over a tested 16-day period at room temperature (FIGS. 10A-10C). The drug release from the nanocarriers was evaluated using dialysis under sink conditions in PBS (pH=7.4) at 37° C. According to the obtained profile, the developed nanocarriers released 40% and 73% of the IRAK 4 inhibitor after 6 and 16 hours, respectively (FIG. 11).

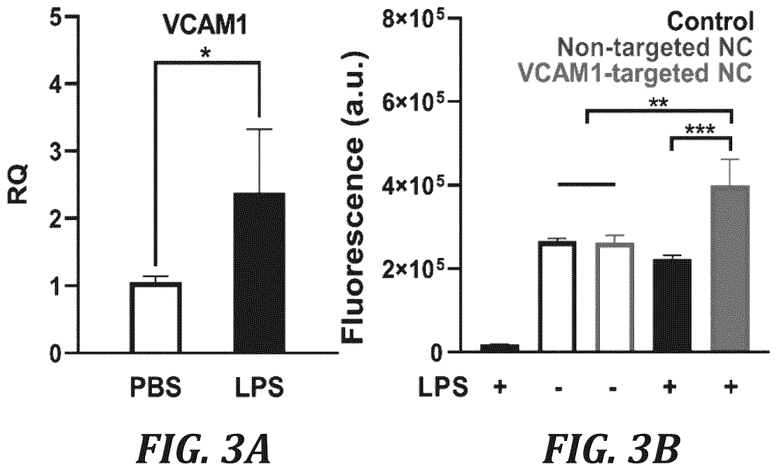

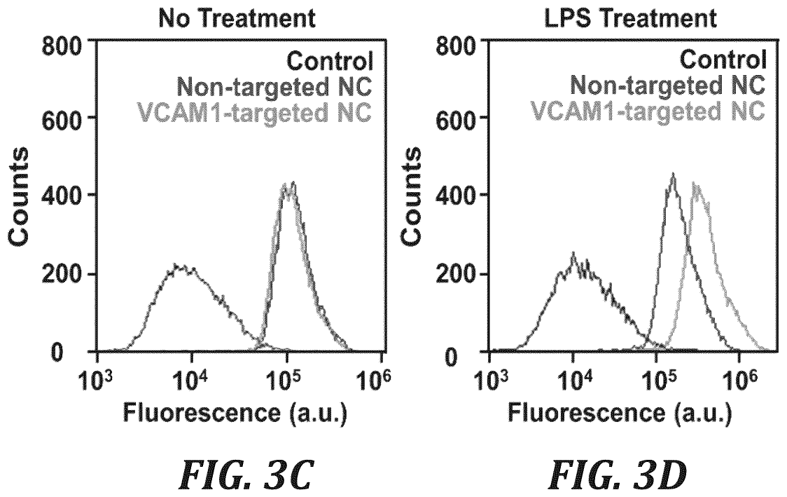

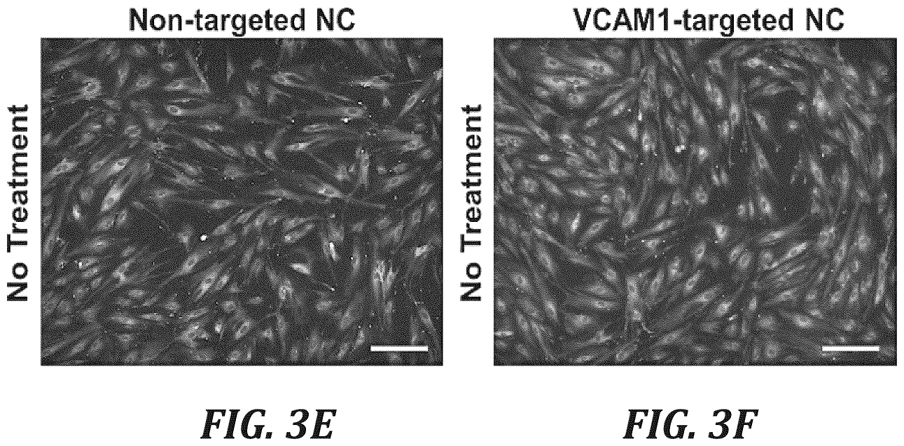

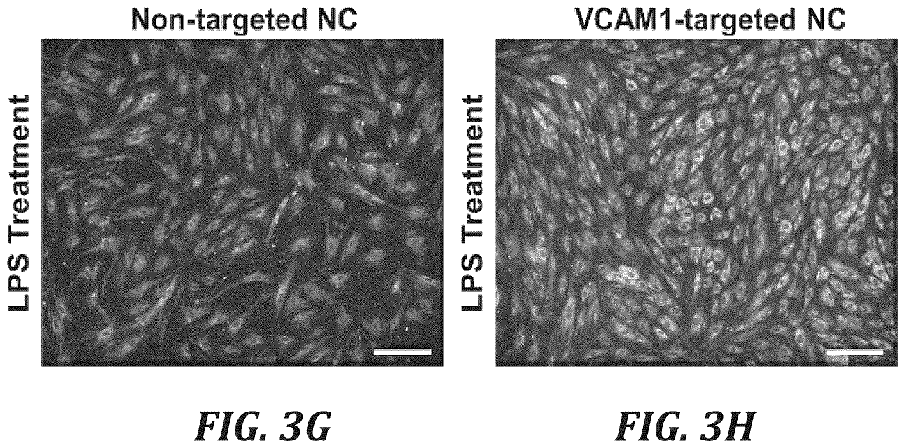

To assess the capacity of VACM1 peptides to increase the internalization efficiency of nanocarriers into inflamed cells overexpressing VCAM1, human colon fibroblast CCD-18Co cells were incubated with 2 μg mL⁻¹ of lipopolysaccharide (LPS) for 24 hours to induce Vcam1 expression. LPS, a major component of the cell wall of gram-negative bacteria, interacts with TLR4 and initiates inflammatory cascades. Vcam1 mRNA levels were found to be 2.4 times higher in LPS-treated cells than in control cells (FIG. 3A).

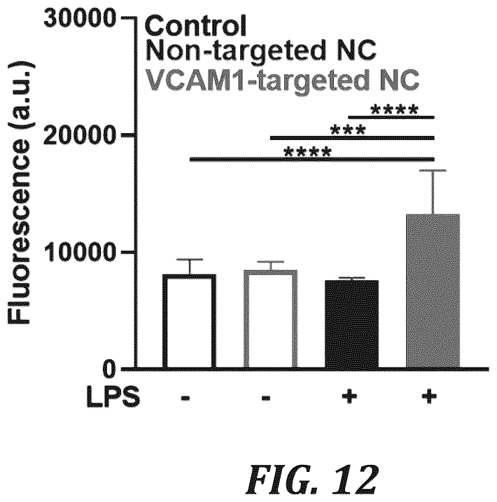

LPS-treated (24 hours pre-treatment) and non-treated CCD-18Co cells were incubated with VCAM1-targeted and non-targeted NCs labeled with Nile Red (a fluorescent dye) for 24 hours and cellular uptake was examined with flow cytometry and fluorescence microscopy. The data show that VCAM1-targeted NCs have superior internalization relative to non-targeted NCs in LPS-treated cells (FIGS. 3B-3H and 12). The mean fluorescent intensity of LPS-treated cells incubated with VCAM1-targeted NC was 1.8 times higher when compared to non-targeted NC (FIGS. 3B and 12). The fluorescence intensities of cells that were not exposed to LPS, on the other hand, did not show a difference between non-targeted NC and VCAM1-targeted NC, and were similar to non-targeted NC in LPS-treated cells. The results reveal that the VCAM1 targeting moiety on the NC surface significantly enhances NC internalization to the inflamed cells.

### In Vivo Toxicity Studies

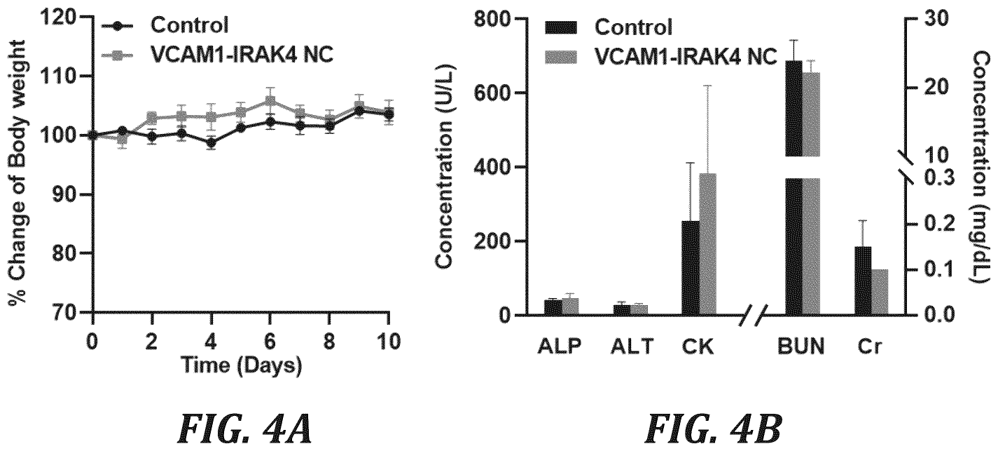

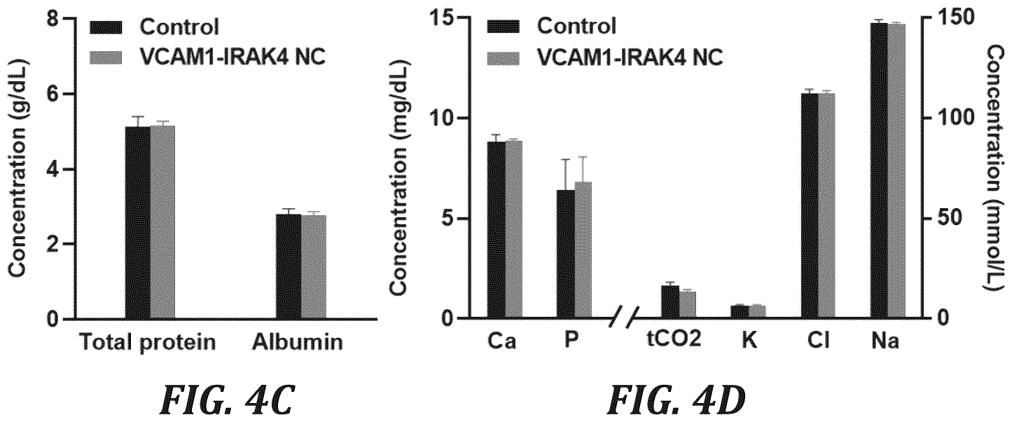

Systemic toxicity of the IRAK4 inhibitor-loaded nanocarriers was evaluated in mice that were injected intravenously (IV) every other day with VCAM1-IRAK4 NC (23 mg kg⁻¹ of PF-06650833) for a total of four injections. Control mice were treated with saline using the same dosage schedule. Because body weight is one of the important considerations for assessment of xenobiotics reactogenicity, mouse body weight was tracked to account for any reactogenic manifestations after VCAM1-IRAK4 NC administration. The mice exhibited no body weight loss in the VCAM1-IRAK4 NC treated group relative to control (FIG. 4A). The biochemical indicators for liver function (alkaline phosphatase (ALP) and alanine aminotransferase (ALT)), kidney function (creatinine (Cr)) and blood urea nitrogen (BUN)) and muscle and heart function (creatine kinase (CK)) in VCAM1-IRAK4 NC treated mice were comparable to those in the control group (FIG. 4B). The biochemistry analysis also revealed that electrolyte and protein concentrations in serum were not different between control and VCAM1-IRAK4 NC injected mice (FIGS. 4C and 4D).

### In Vivo Evaluation of IRAK4 NC and VCAM1-IRAK4 NC in Mice with λ-Carrageenan-Induced Paw Edema

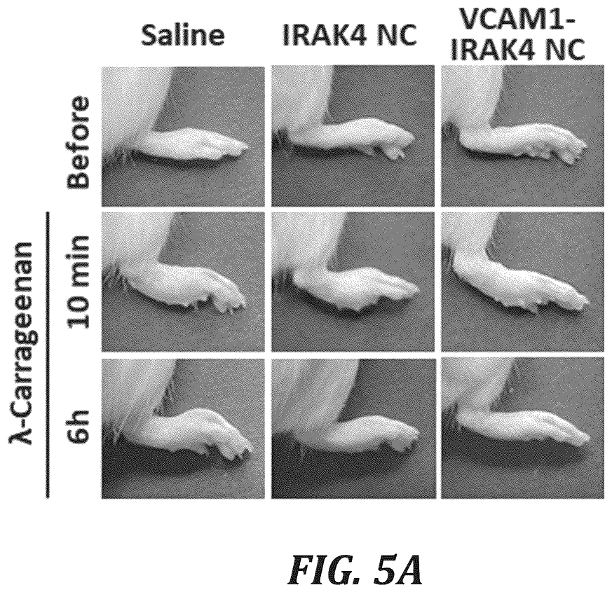

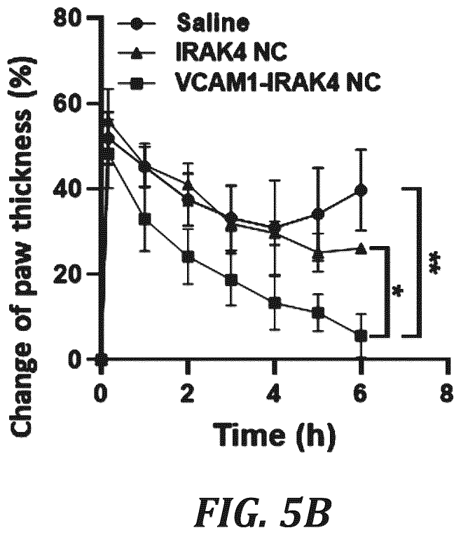

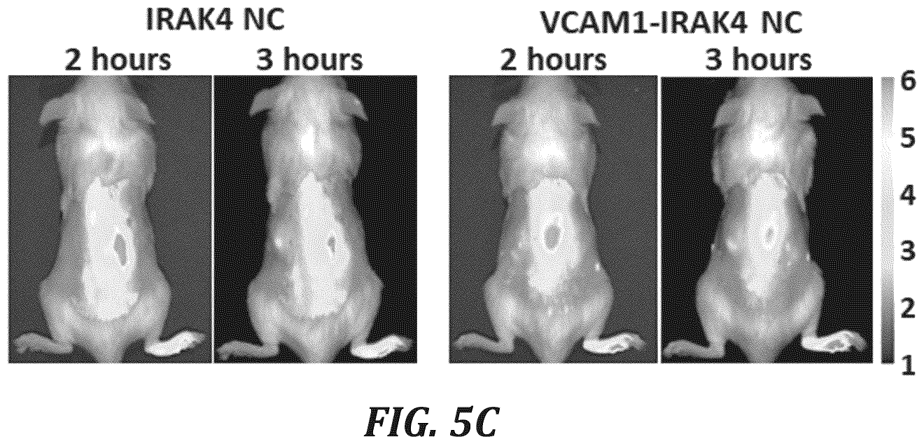

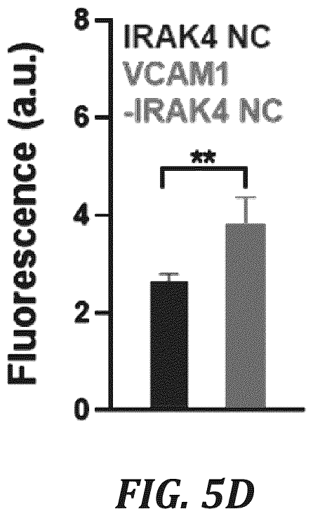

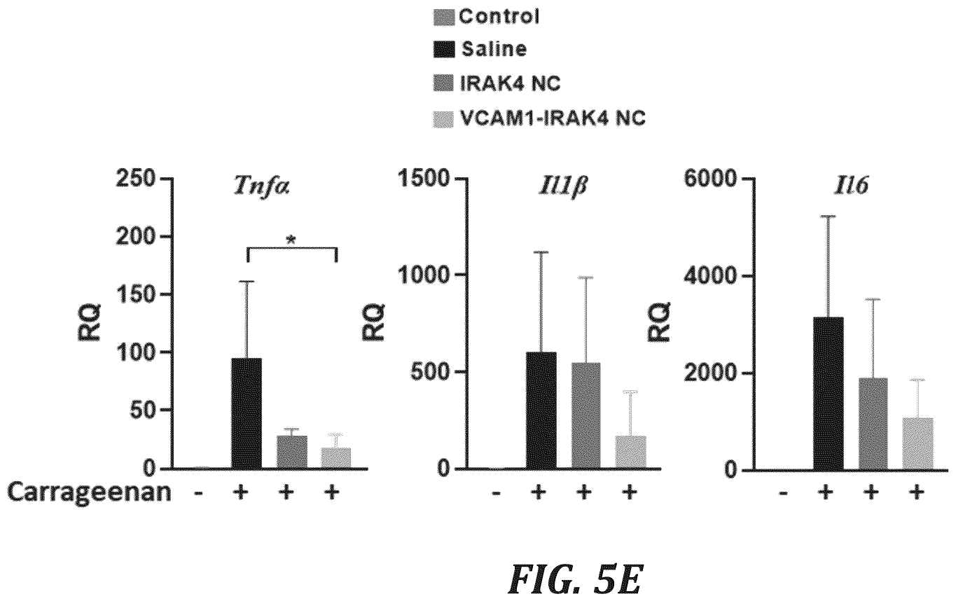

The targeting and anti-inflammatory efficiency of the IRAK4 NC and VCAM1-IRAK4 NC were first evaluated in λ-carrageenan-induced paw edema as an acute local inflammation mouse model. This well-established and reproducible model is widely used to investigate the acute inflammatory response and test new anti-inflammatory drugs. In order to induce inflammatory paw edema in mice, 50 μL of 1% λ-carrageenan, an inflammatory polysaccharide, was injected intraplantarly into the right hind paw. As edema started to develop, paw thickness increased by 50% in the first 10 minutes following λ-carrageenan injection (FIGS. 5A and 5B).

The accumulation of the developed nanocarriers in the inflamed paw following IV injection was assessed by measuring the fluorescence signals from a near-infrared fluorescence dye, silicon 2,3-naphthalocyanine bis (trihexylsilyloxide) (SiNc) co-loaded with IRAK4 inhibitor in PEG-PCL nanoparticles. Whole-body fluorescence images demonstrated that IRAK4 NC and VCAM1-IRAK4 NC after systemic administration accumulated specifically in the inflamed paw when compared to the non-inflamed one in the same mouse (FIG. 5C). Inflammatory processes are associated with loss of endothelial integrity, angiogenesis, and vessel remodeling, all of which enhance vascular permeability in inflamed tissues. Therefore, passive targeting is achieved through the NCs in inflamed targeted tissues, particularly during instances of heightened inflammation and greater penetration of the endothelial barrier. The results provided in FIG. 5C demonstrate that both IRAK4 NC and VCAM1-IRAK4 NC can efficiently accumulate in inflamed paws via passive targeting. Modifying nanoparticles with molecules that bind specifically to receptors overexpressed on target cells (active targeting) can further improve the accumulation of passively delivered nanoparticles in diseased tissues by facilitating their internalization into these cells. Active targeting is a complementary approach to passive targeting, and nanoparticles carrying targeting molecules have to first reach tissue expressing targeted receptors through passive targeting in order to benefit from targeting ligands. To explore the active targeting strategy for the nanocarriers described herein, the nanocarrier surface was modified to present the VHPKQHRGGSKGC (SEQ ID NO: 1) peptide, which has a high affinity for the VCAM1 receptor overexpressed by inflamed endothelial cells. Previous in vitro and in vivo studies demonstrated that nanoparticles containing this peptide preferentially bind to and are internalized by inflamed cells. For the nanocarriers described herein modified on their surface to present the VHPKQHRGGSKGC (SEQ ID NO: 1) peptide, the accumulation of these nanocarriers in inflamed tissue was enhanced. Fluorescence signals in the inflamed paw of mice injected intravenously with VCAM1-IRAK4 NC were increased by 1.5-fold compared to non-targeted nanocarriers (FIG. 5D). These results are supported by in vitro findings indicating that the internalization efficiency of VCAM1-targeted NC in LPS-treated cells was 1.8 times higher than that of non-targeted NC. It will be appreciated that the nanocarriers described herein modified to present the VHPKQHRGGSKGC (SEQ ID NO: 1) peptide may modified with other targeting agents (moieties) that target VCAM1 and other receptors overexpressed on inflamed cells to improve their active targeting efficiency.

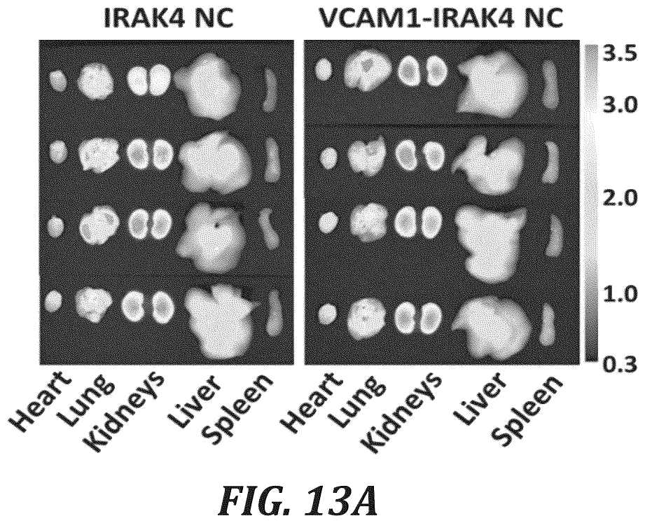

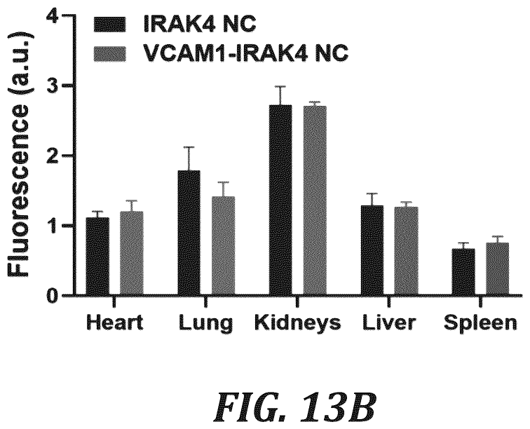

To assess the distribution of IRAK4 NC and VCAM1-IRAK4 NC after systemic administration in tissues other than inflamed paws, major organs were resected and imaged ex vivo. FIGS. 13A and 13B demonstrate that both non-targeted and VCAM1-targeted nanocarriers accumulate substantially in the kidneys and, to a lesser extent, in other organs, including the liver, lungs, and spleen.

To assess the therapeutic efficacy of the prepared formulations, IRAK4NC and VCAM1-IRAK4 NC (11.5 mg kg⁻¹ IRAK4 inhibitor) were administered IV to mice one hour prior to injection of λ-carrageenan and the paw thickness was measured for 6 hours (FIGS. 5A and 5B). These in vivo studies followed previously reported treatment protocols for paw edema model (R. M. Giner, M. L. Villalba, M. C. Recio, S. Manez, M. Cerda-Nicolas, J. Rios, Eur. J. Pharmacol. 2000, 389, 243; J. Wang, H. Wang, H. Xu, J. Li, X. Zhang, X. Zhang, RSC Adv. 2022, 12, 6583). In the majority of the reports employing the λ-carrageenan-induced paw edema model, the therapeutic efficacy of anti-inflammatory drugs is assessed within the initial 5-6-hour period following λ-carrageenan administration into the paw. This practice likely stems from the observation that an injection of λ-carrageenan in the mouse paw elicits a biphasic response, with an early inflammatory reaction lasting for approximately 6 hours. Of note, the drug release profile demonstrates that the developed nanocarriers released 40% of the IRAK4 inhibitor within the first 6 hours (FIG. 11). At the 6-hour mark, the paw thickness of mice treated with VCAM1-IRAK4 NCs exhibited only a 5% increase in comparison to the initial thickness of the same paw prior to inflammation (FIG. 5B). The change in paw swelling in the control group, injected with saline, remained around a 40% increase at 6 hours, while the paw thickness of mice administered with non-targeted IRAK4 NC was 26%. (FIG. 5B).

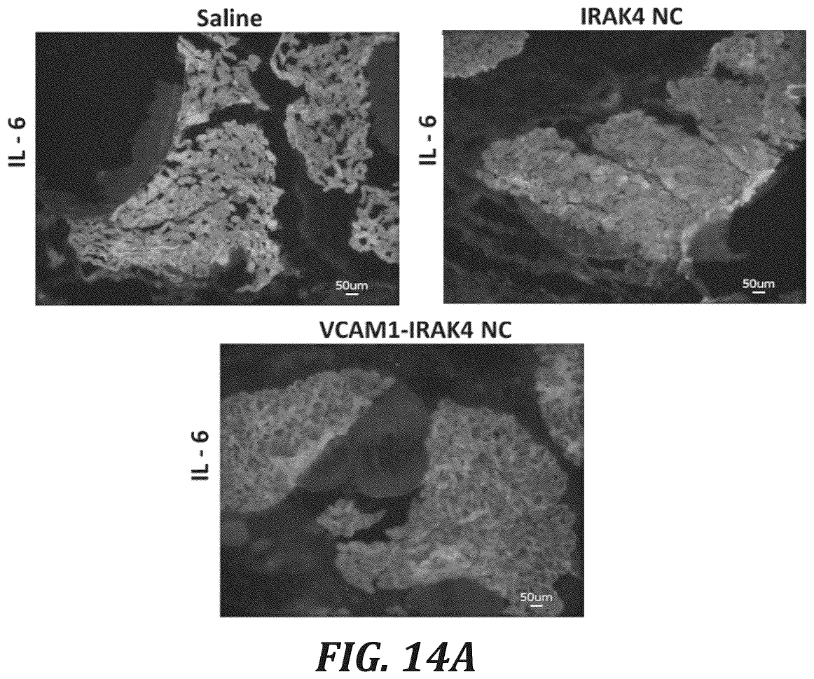

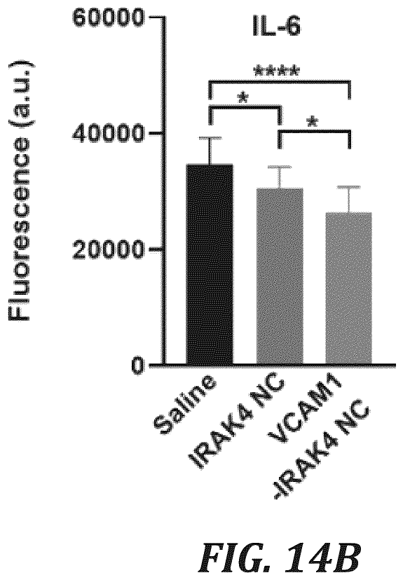

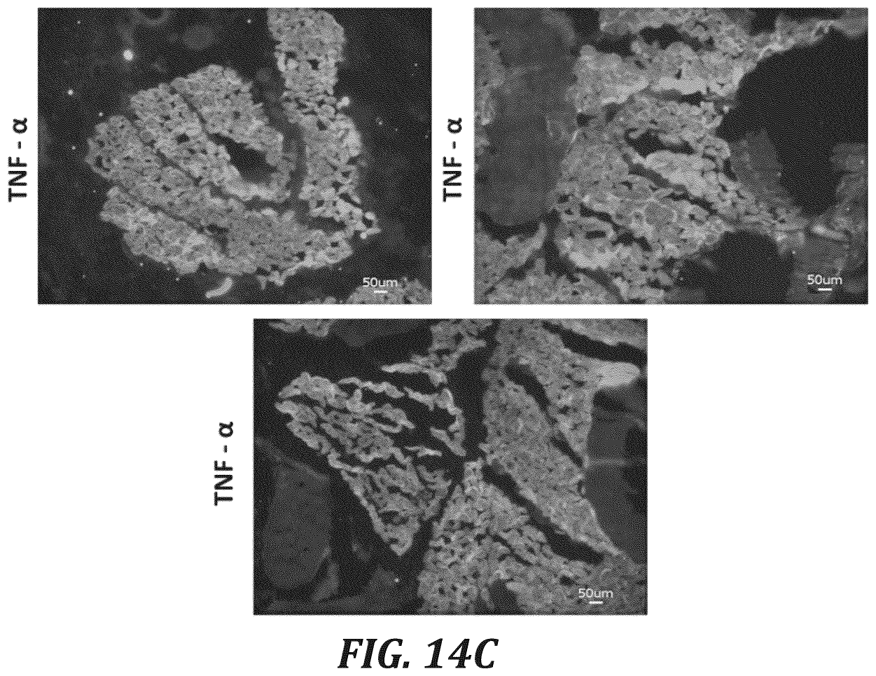

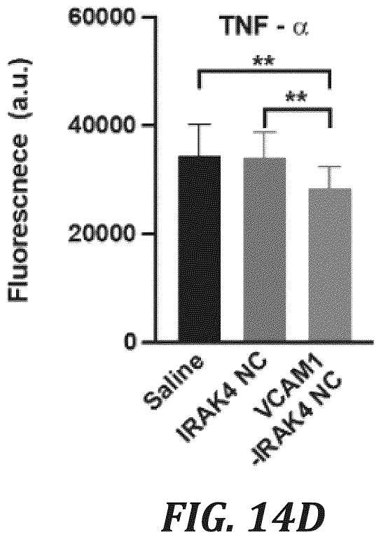

The mRNA levels of pro-inflammatory cytokines, Tnf, Il1β, and Il6 in paws was measured (FIG. 5E). When compared to the negative group (no λ-carrageenan injection), λ-carrageenan administration led to a significant upregulation of Tnf, Il1β, and Il6 mRNA expression in the paws. Both IRAK4 NC and VCAM1-IRAK4 NC treated groups displayed lower levels of cytokines than the saline treated group. In comparison to IRAK4 NC, VCAM1-IRAK4 NC remarkably reduced the levels of Tnf, Il1β, and Il6. Immunofluorescence staining experiments also confirmed that IRAK4 NC and VCAM1-IRAK4 NC treated paw edema tissues had lower levels of IL-6 and TNF-α cytokines than saline-treated tissues (FIGS. 14A-14D). Furthermore, cytokine levels in the VCAM1-IRAK4 NC treated tissues were significantly lower when compared to the IRAK4 NC ones. These findings demonstrate that VCAM1-IRAK4 NC markedly abated the inflammation of carrageenan-induced paw edema.

### In Vivo Evaluation of VCAM1-IRAK4 NC in Mice with DSS-Induced Colitis

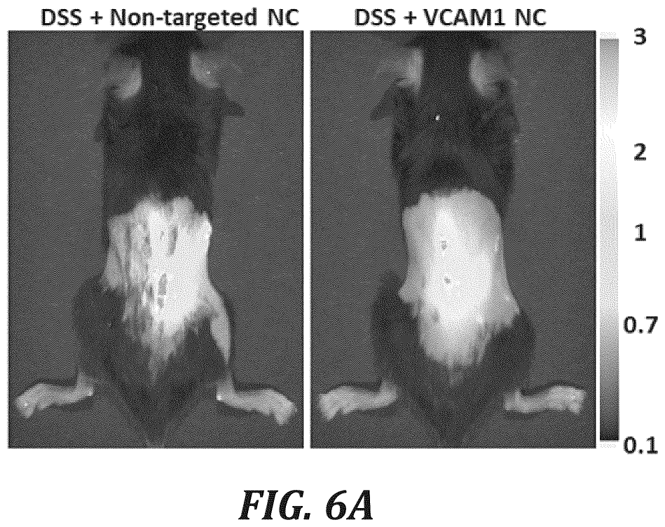

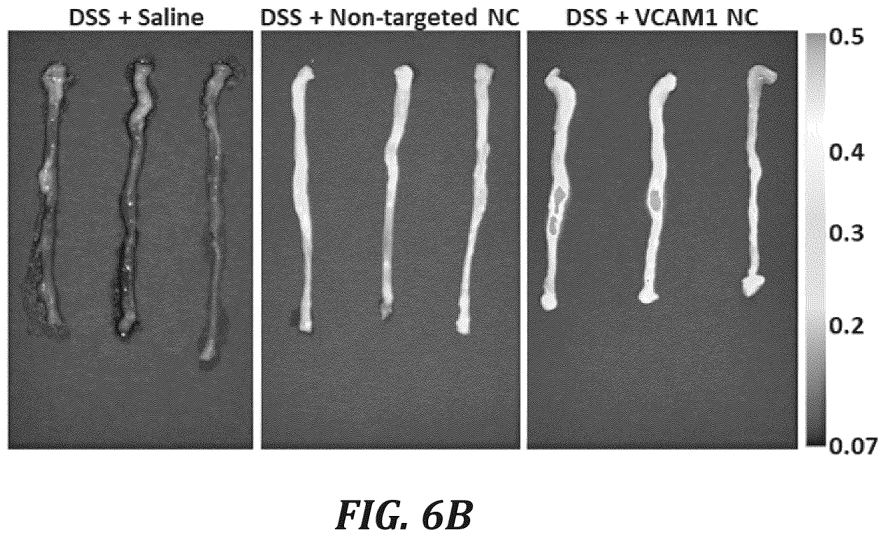

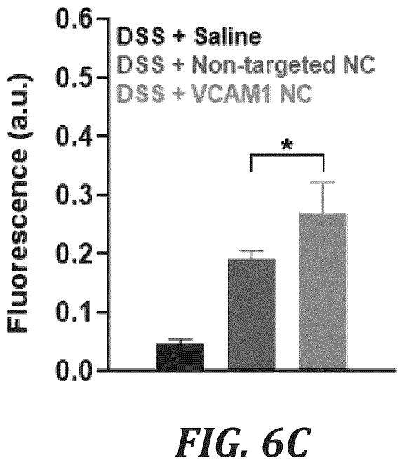

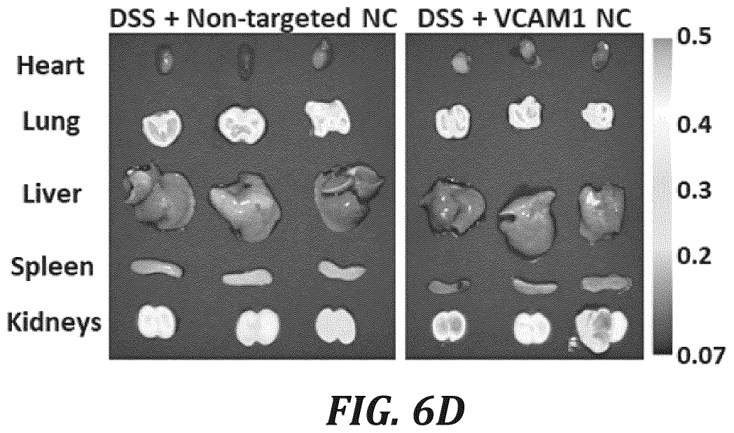

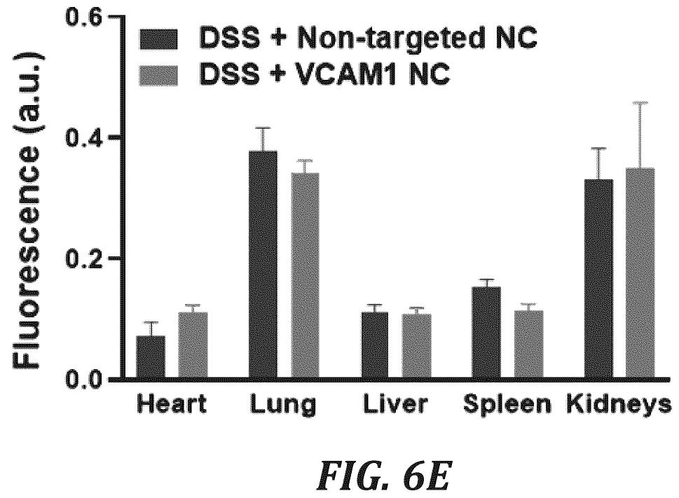

Encouraged by the therapeutic effect on paw edema, the dextran sulfate sodium (DSS)-induced colitis model was exploited to further validate the anti-inflammatory efficacy of the VCAM1-IRAK4 NC formulation. Colon inflammation was induced in mice by administering 5% DSS in drinking water for 7 consecutive days. To evaluate the biodistribution of non-targeted and VCAM1-targeted nanocarriers, mice with induced colitis were injected IV with SiNc-loaded IRAK4 NC and VCAM1-IRAK4 NC, and NIR fluorescence images of the shaved mice, excised colons and major organs were recorded 24 hours post-administration (FIGS. 6A-6E). The results indicated that the mean fluorescence intensity of SiNc in inflamed colons treated with VCAM1-targeted nanocarriers was 1.4 times higher when compared to non-targeted ones (FIGS. 6B and 6C). These findings are supported by a previous study showing that VCAM1 is overexpressed in colonic endothelium of mice with DSS-induced colitis threefold more than in control mice. Ex vivo images of major organs reveal that both non-targeted and VCAM1-targeted nanocarriers also accumulate in the lungs and kidneys and, to a lesser degree, in the liver, spleen, and heart (FIGS. 6D and 6E).

To evaluate the anti-inflammatory action of the developed VCAM1-IRAK4 NC, mice were administered with 5% DSS in drinking water for 7 days and injected IV with the VCAM1-targeted nanocarriers loaded with IRAK4 inhibitor (23 mg kg⁻¹) on day 0, 2, 4, and 6 of DSS treatment initiation (Y. Lec, K. Sugihara, M. G. Gillilland, 3rd, S. Jon, N. Kamada, J. J. Moon, Nat. Mater. 2020, 19, 118). The drug release profile suggests that the nanocarriers described herein released over 90% of the IRAK4 inhibitor within 2 days (FIG. 11). A healthy control group without DSS administration and a DSS-induced colitis group without the injection of VCAM1-IRAK4 NC were also prepared. The IRAK4 inhibitor, PF-06650833, has not previously been tested for the treatment of colitis. Therefore, a reasonable dose (23 mg kg⁻¹) was selected for the study based on available publications where this drug was evaluated for other inflammation disorders: Yoon et al. used oral doses of PF-06650833 at 50 and 100 mg kg⁻¹ to treat LPS-mediated systemic inflammation in mice (S. B. Yoon, H. Hong, H. J. Lim, J. H. Choi, Y. P. Choi, S. W. Seo, H. W. Lee, C. H. Chae, W. K. Park, H. Y. Kim, D. Jeong, T. Q. De, C. S. Myung, H. Cho, Acta Pharm. Sin. B 2023, 13, 1093), while Li et al. administered an oral dose of 100 mg kg⁻¹ to mice with acute respiratory distress syndrome (Q. Li, R. Li, H. Yin, S. Wang, B. Liu, J. Li, M. Zhou, Q. Yan, L. Lu, Biomed. Pharmacother. 2022, 153, 113459).

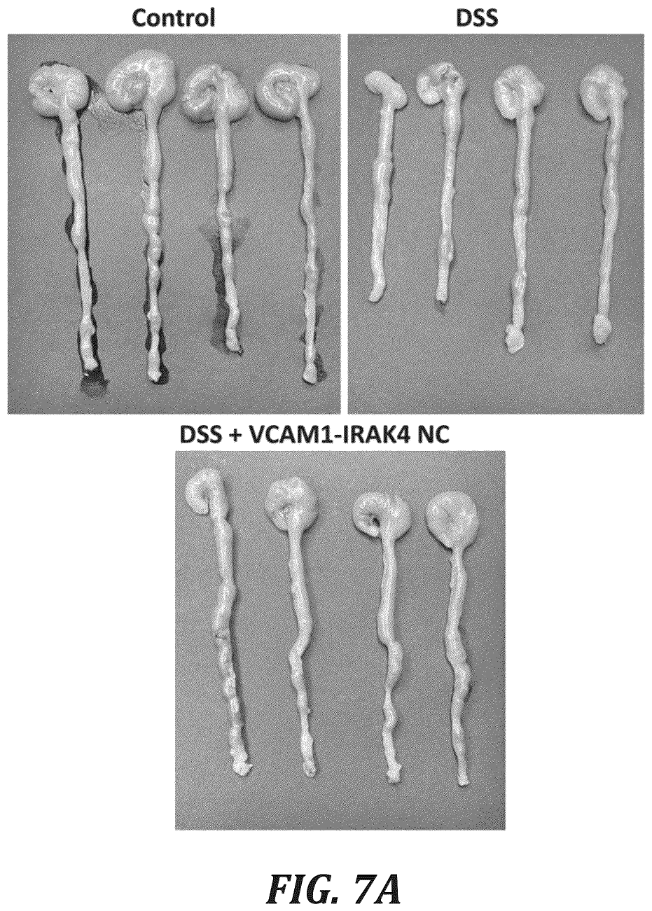

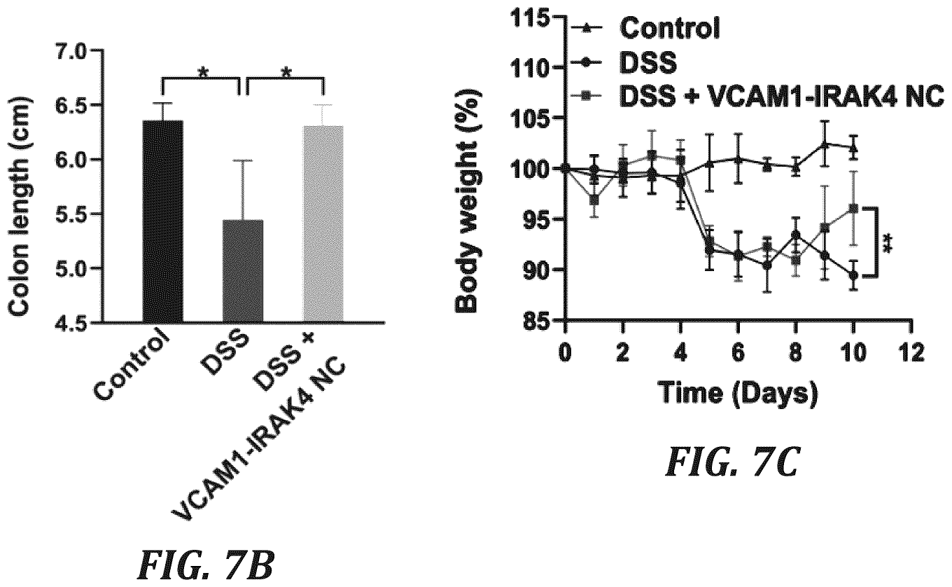

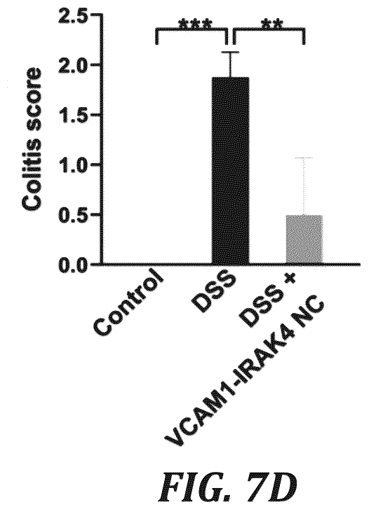

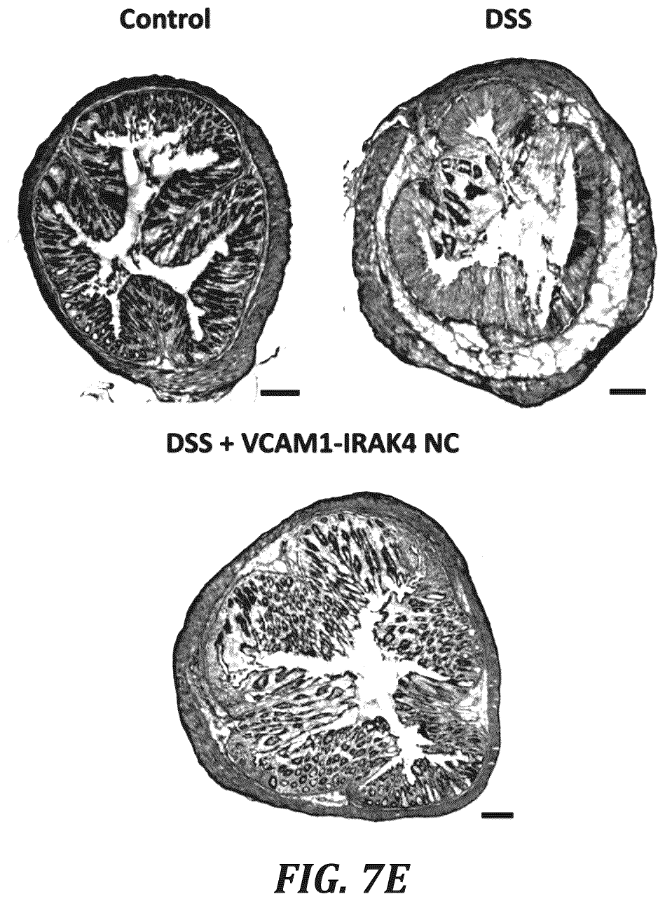

On day 10, mice were euthanized, their entire colons were excised, and colon length reduction was assessed as a key indicator of the severity of DSS-induced colitis. The average colon length of healthy mice was 6.35±0.16 cm, whereas this parameter decreased significantly to 5.44±0.55 cm in animals with DSS-induced colitis (FIGS. 7A and 7B). Remarkably, the colon length of colitis mice treated with VCAM1-IRAK4 NC was 6.31±0.19 cm, which was not statistically different from that of the healthy control group, indicating a reduction in inflammation. In addition to colon length shortening, weight loss is a typical symptom of colitis, which can be caused by a lack of appetite, decreased nutrient and water absorption and other factors. As shown in FIG. 7C, the body weight of mice with DSS-induced colitis decreased drastically to 89% of their initial body weight, whereas animals injected with VCAM1-IRAK4 NC experienced a similar decrease in body weight but recovered to 96% of their initial body weight after treatment.

Colitis-dependent damage was assessed in H&E-stained colon sections to further examine the therapeutic effect of VCAM1-IRAK4 NCs on the local colonic environment (FIGS. 7D and 7E). In the histological analysis, the colon tissues of mice with DSS-induced colitis exhibited clear inflammatory signs, such as abnormal mucosal structure and significant colonic crypt distortion, when compared to those of the healthy control group. In contrast, the colons of colitis mice treated with VCAM1-IRAK4 NC showed a significant decrease in inflammation, including regeneration of thick colonic epithelium and crypt structure similar to the healthy control group. A board-certified pathologist utilized blinded analysis to score colonic damage using a three-point scale corresponding to no, mild/moderate, or severe inflammation (scoring range: 0 to 2). The healthy control group and the DSS-induced colitis group had colon damage scores of 0 and 1.88, respectively (FIG. 7D). Notably, in comparison to the saline treated colitis group, the VCAM1-IRAK4 NC treated group exhibited a relatively normal colon damage score of 0.5, which implies a significant anti-inflammatory effect of the developed therapeutic.

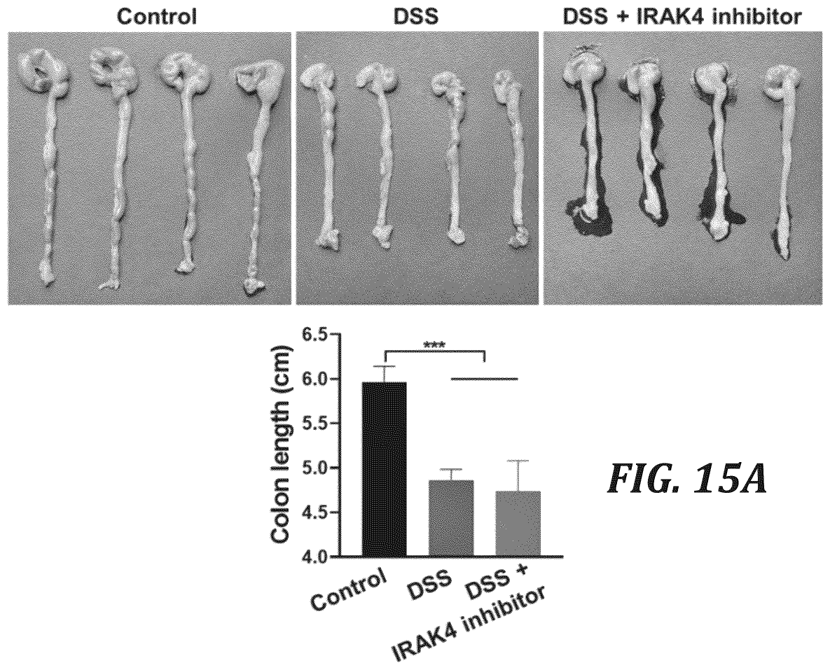

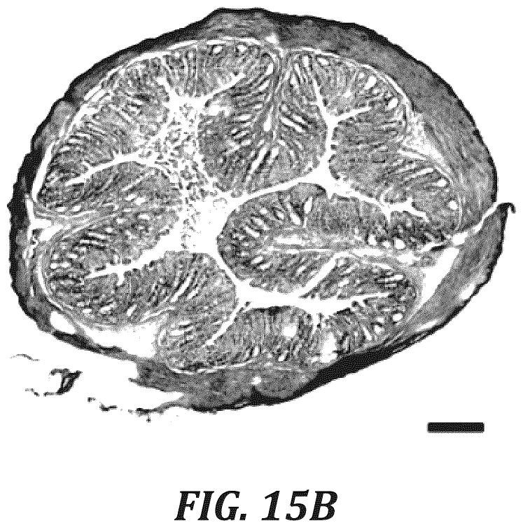

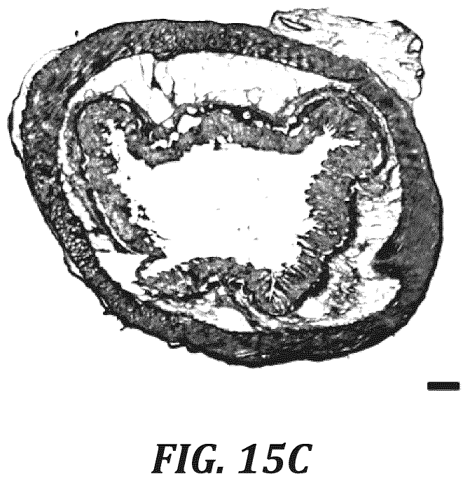

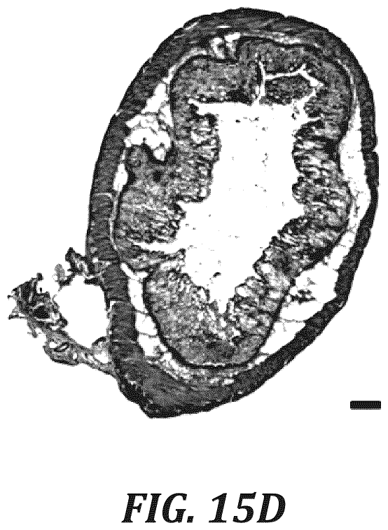

To demonstrate the significance of the developed nanocarriers, the therapeutic efficacy of a free IRAK4 inhibitor in mice with DSS-induced colitis was evaluated. Due to its limited aqueous solubility (57 μg mL⁻¹), the drug was formulated in a mixture of 60% saline, 30% PEG400 and 10% DMSO to achieve the required therapeutic dose. In contrast to the IRAK4 inhibitor loaded in VCAM1-targeted nanocarriers (FIGS. 7A-7E), a free drug administered at the same dosage regimen (23 mg kg⁻¹, 4 IV injections every 2 days) exhibited no noticeable therapeutic effect on the treatment of colitis in mice (FIGS. 15A-15D). For example, the average colon length of mice treated with a free IRAK4 inhibitor (4.74±0.35 cm) was not statistically different from that of colitis animals injected with saline (4.87±0.12 cm), but it was substantially shorter than that of healthy mice (5.97±0.18 cm) (FIGS. 15A-15D).

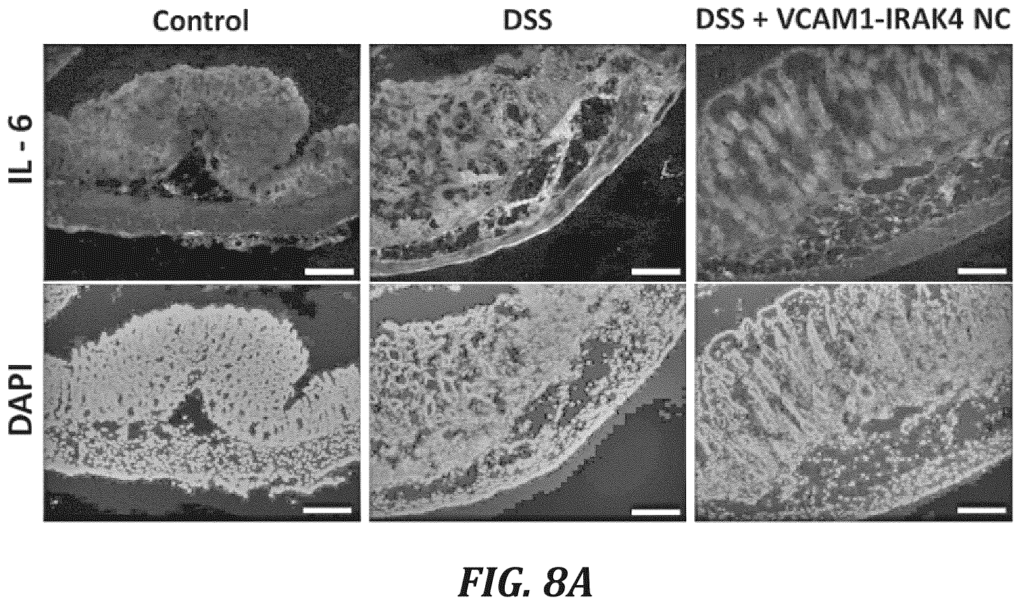

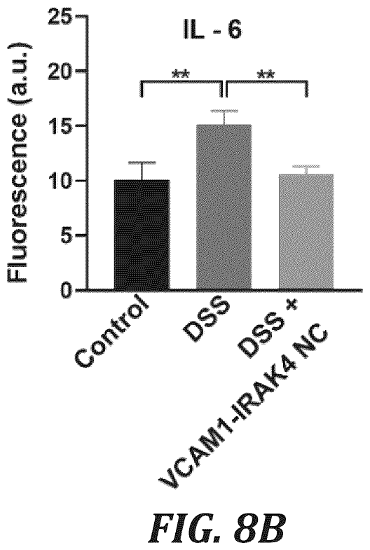

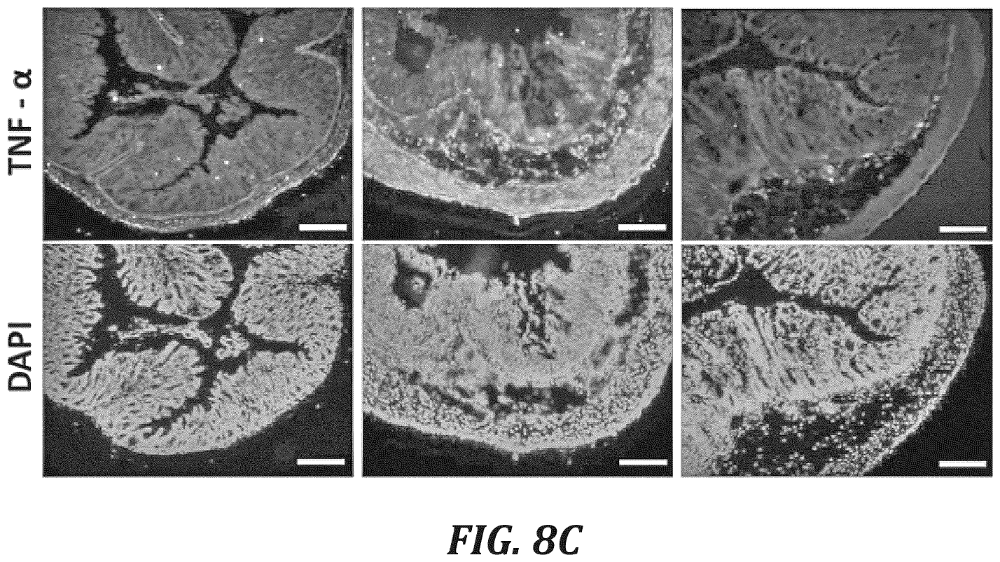

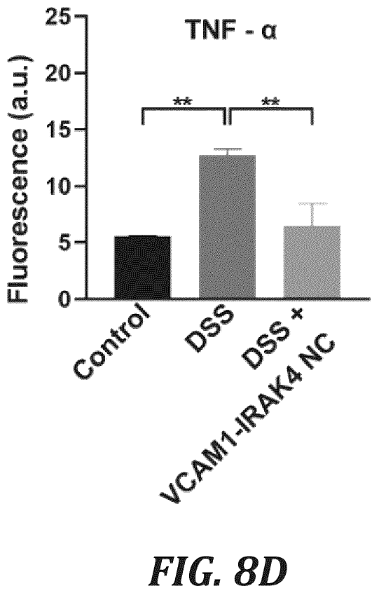

Immunofluorescence staining of collected colon tissues was also performed to visualize the protein expression of IL-6 and TNF-α. As shown in FIG. 8A, the inflamed tissues of the saline treated colitis group (DSS) showed strong staining for IL-6 and TNF-α. These cytokines were expressed in practically every region, including mucosa, submucosa, and smooth muscle, which is consistent with previous findings in DSS-induced colitis. However, the DSS-induced colitis group treated with VCAM1-IRAK4 NC had low fluorescence signals of pro-inflammatory cytokines, similar to the healthy control group.

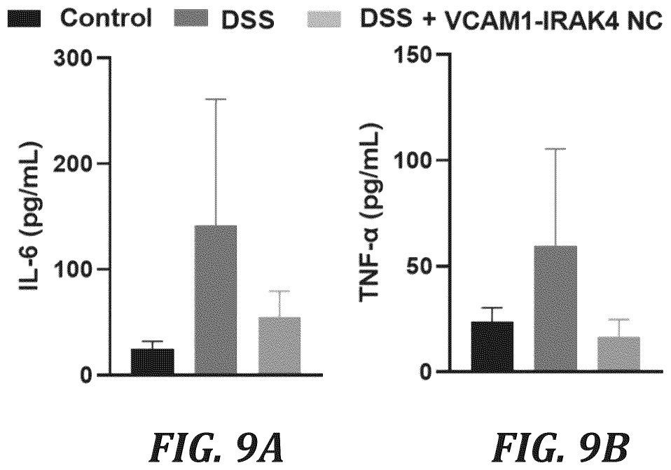

Finally, ELISA analysis was performed to compare the expression levels of pro-inflammatory cytokines throughout the whole colon (FIGS. 9A and 9B). In the DSS-induced colitis group, IL-6 and TNF-α levels were considerably higher than in control healthy colons. However, the VCAM1-NC treated group had significantly lower IL-6 and TNF-α levels than the DSS-induced colitis group.

In summary, the VCAM-1-targeted nanocarriers described herein dramatically increase the aqueous dispersibility of a clinically-tested IRAK 4 inhibitor and efficiently accumulate in the inflamed tissues of mice with induced paw edema and colitis after intravenous administration. Systemically administered VCAM1 IRAK4 NC exhibited a significant anti-inflammatory effect by reducing carrageenan-induced paw edema and attenuating colitis manifestations such as body weight loss, colon shrinkage, and colonic tissue damage. This was mirrored by a decrease in the levels of pro-inflammatory cytokines in the affected tissues of treated mice. Importantly, repeated VCAM1-IRAK4 NC injections do not induce any acute side effects in the major organs, including the liver, kidney, muscle, and heart. The efficacy of targeted IRAK4 inhibition in regulating tissue inflammatory responses, as shown by the results described herein, demonstrates the application of this nanomedicine strategy in the treatment of various inflammatory diseases with minimal adverse effects.

As used herein, the term “about” refers to ±5% of the specified value.

### Materials/Methods

Materials. The methoxy PEG-PCL (CH: O-PEG-PCL) with molecular weight PEG(5k)-PCL(10k)) was obtained from Advanced Polymer Materials Inc. (Montreal, Canada). The IRAK4 inhibitor (PF06650833), lipopolysaccharide (O111: B4; LPS), and maleimide PEG-PCL with molecular weight MAL-PEG(5k)-PCL(10k)) were purchased from Sigma Aldrich (St. Louis, MO, USA). The silicon 2,3-naphthalocyanine bis (trihexylsilyloxide) was obtained from Alfa Chemistry (Ronkonkoma, NY, USA). VCAM1 peptide (VHPKQHRGGSKGC, (SEQ ID NO: 1)) was obtained from Biomatik (Wilmington, DE, USA). The Measure-iT™ Thiol Assay Kit was obtained from Invitrogen (Waltham, MA, USA). Nile Red was obtained from MP Biomedicals (Irvine, CA, USA). Dextran Sulfate Sodium with a molecular weight of about 40 kDa was purchased from TdB Labs (Uppsala, Sweden), and low viscosity λ-Carrageenan was obtained from TCI America (Portland, OR, USA).

PEG-PCL polymer conjugation with VCAM1 targeting peptide. A mixture of maleimide PEG-PCL (32.0 mg, 2.1 μmol) in acetonitrile (1.5 mL) and VCAM1 peptide (3.2 mg, 2.3 μmol) in dimethyl sulfoxide (150.0 μL) was incubated at room temperature overnight. The conjugation was confirmed using the Measure-iT™ Thiol Assay Kit as per the manufacturer's instructions. Specifically, the resultant product fluorescence was measured upon mixing with the thiol-reactive fluorescent detector (517 nm emission, 494 nm excitation). The thiol-reactive reagent selectively labeled the free thiol group of unconjugated peptides. The yield of the conjugation reaction was found to be 96.5%.

Preparation and characterization of VCAM1-targeted and non-targeted IRAK4 inhibitor-loaded nanocarriers. The solvent evaporation approach was used to prepare VCAM-targeted and non-targeted nanocarriers loaded with an IRAK4 inhibitor (designated as IRAK4 NC and VCAM1-IRAK4 NC, respectively) (Y. Park, A. A. Demessie, A. Luo, O. R. Taratula, A. S. Moses, P. Do, L. Campos, Y. Jahangiri, C. R. Wyatt, H. A. Albarqi, K. Farsad, O. D. Slayden, O. Taratula, Small 2022, 18, e2107808; F. Y. Sabei, O. Taratula, H. A. Albarqi, A. M. Al-Fatcase, A. S. Moses, A. A. Demessie, Y. Park, W. K. Vogel, E. E. Nazzaro, M. A. Davare, A. Alani, M. Leid, O. Taratula, Nanomed. Nanotechnol. Biol. Med. 2021, 102446). Specifically, for the preparation of non-targeted IRAK4 NC, a solution of 1.0 mL of methoxy PEG-PCL (100.0 mg mL⁻¹) in tetrahydrofuran (THF) and 1.5 mL of IRAK4 inhibitor (6.0 mg mL⁻¹) in acetone was combined. Subsequently, 2.0 mL of saline was delivered to the reaction solution and stirred for 1 minute. The resulting solution was then allowed to stand at room temperature overnight to evaporate THF and acetone. For the preparation of VCAM1-IRAK4 NC, a mixture of VCAM1-PEG-PCL and CH₃O-PEG-PCL with a 1:9 ratio in THF was used in the first step of the procedure. The obtained aqueous solution was centrifuged at 10,000 rpm for 1 min, and the supernatant was filtered through a 0.2 μm nylon filter to remove any possible aggregates. The morphology, hydrodynamic size, polydispersity index, and surface charge of the resulting IRAK4 NC and VCAM1-IRAK4 NC were evaluated by following our previously reported methods (A. A. Demessie, Y. Park, P. Singh, A. S. Moses, T. Korzun, F. Y. Sabei, H. A. Albarqi, L. Campos, C. R. Wyatt, K. Farsad, P. Dhagat, C. Sun, O. R. Taratula, O. Taratula, Small Methods 2022, e2200916).

The amount of IRAK4 inhibitor loaded into polymeric nanocarriers was quantified using a Shimadzu high performance liquid chromatography (HPLC) system equipped with a Zorbax C18 column (Santa Clara, CA, USA). A standard curve was generated by eluting known amounts of serially diluted IRAK4 inhibitor samples ranging from 0.0375 mg mL⁻¹ to 0.3 mg mL⁻¹. Standard samples of known concentrations were prepared by dissolving IRAK4 inhibitor in acetone. The elution process was carried out at a flow rate of 0.2 mL min⁻¹ using an isocratic method with a mobile phase consisting of 60% (v/v) acetonitrile in water containing 0.1% trifluoroacetic acid. The IRAK4 inhibitor peak was detected at 350 nm and the peak area was measured for each standard solution. To determine the amount of IRAK4 inhibitor loaded in polymeric nanocarriers, samples of IRAK4-loaded nanocarriers were diluted 30 times with acetone and analyzed using the same HPLC method. The amount of IRAK4 inhibitor loaded into the polymeric nanocarriers was calculated using the equation derived from a standard curve, relating the peak area to the concentration of the IRAK4 inhibitor. Considering the dilution factor of 30, it was determined that the loaded amount of IRAK4 inhibitor in nanocarriers is 4.59 mg. The analyzed samples of IRAK4-loaded nanocarriers were prepared by mixing 6.0 mg of the IRAK4 inhibitor with PEG-PCL. The encapsulation efficiency (%) was calculated as amount of IRAK4 inhibitor loaded in nanocarriers (mg)/initial amount of IRAK4 inhibitor (mg)×100. Therefore, the encapsulation efficiency of the IRAK4 inhibitor in the nanocarrier is 4.59 mg/6.0 mg×100=76.5%.

For the drug release study, IRAK4-loaded NC in PBS was loaded into a dialysis membrane with a 3 kDa molecular cutoff. The loaded membrane was then immersed in PBS and stirred at 37° C. to maintain physiological conditions. At predetermined time intervals, 1 mL aliquots of the release medium were withdrawn from the external compartment surrounding the dialysis membrane and added 1 mL PBS. The concentration of IRAK inhibitor in each aliquot was measured using the above described HPLC method.

To conduct the biodistribution study, SiNc-loaded VCAM1-IRAK4 NC and IRAK4 NC were prepared. To achieve this, SiNc (0.15 mg) was delivered to the above-described mixture of PEG-PCL and IRAK4 inhibitor.

In vitro studies. Human colon fibroblast CCD-18Co cells were purchased from ATCC (Manassas, VA, USA). CCD-18Co cells were seeded in six-well plates at a density of 30,000 cells per well. Cells were then incubated with LPS (2 μg mL⁻¹) or PBS in 1% bovine serum albumin (BSA) medium. After 24 hours, the medium was removed, and cells were washed with DPBS three times. Non-targeted and VCAM1-targeted NC labeled with Nile Red were added to the medium and incubated with cells for 24 hours. Subsequently, the medium was eliminated, and the cells were washed three times with PBS before being harvested. The fluorescence produced by the internalized Nile Red within the cells was quantified using a BD Accuri C6 flow cytometer (BD Biosciences, CA, USA). Cells that had not been incubated with NC (medium only) served as a control. To perform fluorescence microscopy studies, CCD-18Co cells were seeded in six-well plates at a density of 150,000 cells per well cells and treated as described above. Keyence BZ-X700 microscope (Keyence Corp., Osaka, Japan) was used to obtain fluorescence images of cells. The fluorescence signal in the microscopy images was quantified using the ImageJ software. To confirm VCAM1 expression in CCD-18Co cells under the pro-inflammatory conditions, CCD-18Co cells were subjected to 2 μg mL⁻¹ of LPS in combination with endotoxin-free bovine serum albumin or PBS for control group. At 24 hours after treatment, cells were harvested, and VCAM1 qRT PCR was performed using Taqman gene expression primers (Applied Biosystems, San Francisco, CA, USA; Mm01320970_m1) according to the previously reported procedure (T. Korzun, A. S. Moses, J. Kim, S. Patel, C. Schumann, P. R. Levasseur, P. Diba, B. Olson, K. G. O. Rebola, M. Norgard, Y. Park, A. A. Demessie, Y. Eygeris, V. Grigoriev, S. Sundaram, T. Pejovic, J. R. Brody, O. R. Taratula, X. Zhu, G. Sahay, D. L. Marks, O. Taratula, Small 2022, 18, 2204436). The process of normalizing gene expression was conducted with reference to the expression levels of the murine 18s gene (Mm03928990_g1).

In vivo studies. The Institutional Animal Care and Use Committee of Oregon Health and Science University approved all animal studies utilized in the study.

In vivo toxicity studies. To evaluate the potential toxicity of VCAM-1targeted nanocarriers loaded with the IRAK4 inhibitor, VCAM1-IRAK4 NC (PF-06650833 dose: 23 mg kg⁻¹) and saline were administered intravenously to two groups of Swiss Webster mice (four mice per group) four times every other day. Body weight was measured daily for 10 days. Blood samples were taken on Day 10 and the levels of the biomarkers were assessed by the IDEXX laboratory (Portland, OR, USA).

In vivo evaluation of VCAM1-IRAK4 NC in mice with carrageenan-induced paw edema. To establish the paw edema murine model, female Swiss Webster mice were used (Charles River Laboratories, Wilmington, MA, USA). Each group of 3 mice received VCAM1-IRAK NC (PF-06650833 dose: 11.5 mg kg⁻¹), IRAK NC (PF-06650833 dose: 11.5 mg kg⁻¹), or saline intravenously. After 1 hour, paw edema was induced by administration of 50 μL λ-carrageenan (1% w/v in saline) into the plantar surface of the right hind paw except for the control group. For 6 hours, the paw thickness was measured hourly with a digital caliper. The following equation was employed to calculate the percentage increase in paw thickness: Paw thickness increase (%)=(Tₜ−T₀)/T₀×100%, where T₀ and Tₜ represent the paw thickness before and after carrageenan injection, respectively. The mice were then euthanized, and paw tissues were collected and used to analyze mRNA levels of pro-inflammatory cytokines, such as Tnf, Il1β, and Il6. In summary, the RNA extraction from the paws was carried out by utilizing TRIzol (Ambion, Carlsbad, USA) and chloroform, followed by further extraction using Qiagen RNeasy Mini kit (Qiagen Corporation, Hilden, Germany). Proteinase K (Qiagen Corporation, Hilden, Germany) was used for fibrous tissues during the tissue homogenization process. Subsequently, mRNA levels of pro-inflammatory cytokines were measured with qRT-PCR utilizing TaqMan assays targeting Tnf, Il1β, and Il6 (Mm00443258_m1, Mm00434228_m1, Mm00446190_m1) (T. Korzun, A. S. Moses, J. Kim, S. Patel, C. Schumann, P. R. Levasseur, P. Diba, B. Olson, K. G. O. Rebola, M. Norgard, Y. Park, A. A. Demessie, Y. Eygeris, V. Grigoriev, S. Sundaram, T. Pejovic, J. R. Brody, O. R. Taratula, X. Zhu, G. Sahay, D. L. Marks, O. Taratula, Small 2022, 18, e2204436). The normalization of gene expression was accomplished with reference to the expression levels of murine 18S (Mm03928990_g1).

To conduct the biodistribution study, the shaved mice with paw edema were injected IV with SiNc-loaded VCAM1-IRAK4 NC and IRAK4 NC. The fluorescence images were obtained with a Pearl Impulse imaging system (LI-COR, Lincoln, NE) at various time points after administration of nanocarriers, and the mean fluorescence intensity of region of interest was quantified using Pearl Impulse Software (LI-COR, Lincoln, NE). After euthanasia, major organs were collected and imaged, and the fluorescence signal was quantified as described above.

In vivo evaluation of VCAM1-IRAK4 NC in mice with DSS-induced colitis. Ten-week-old female C57BL/6 mice were purchased from Charles River Laboratories (Wilmington, MA, USA) and acclimatized for 1 week prior to DSS treatment. For 7 days, mice received drinking water containing 5% (w/v) DSS, followed by normal water. Healthy control mice were provided with normal water only. To evaluate the accumulation of VCAM1-targeted nanocarriers in inflamed tissue, the shaved mice with induced colitis were injected IV with SiNc-loaded IRAK4 NC and VCAM1-IRAK4 NC, and NIR fluorescence images of the excised colons and major organs were recorded 24 hours post-administration. The fluorescence images were obtained using a Pearl Impulse fluorescence imaging system (LI-COR, Lincoln, NE) and the mean fluorescence intensity of region of interest was obtained using Pearl Impulse Software (LI-COR, Lincoln, NE). For evaluation of the anti-inflammatory action of the developed VCAM1-IRAK4 NC, VCAM1-IRAK4 NC or saline was administered intravenously on days 0, 2, 4, and 6. To assess the therapeutic efficacy of the free IRAK4 inhibitor (without nanocarriers), the drug was formulated in a mixture of 60% Saline, 30% PEG400, and 10% DMSO and administered as described above. The IRAK4 inhibitor dose in all treatment groups was 23 mg kg⁻¹ per injection. During a 10-day experimental period, daily assessments of weight changes were conducted. On day 10, animals were euthanized and their entire colons were resected. Colon length without the cecum was measured, and colon tissues were gently washed with PBS. The colon tissues were frozen in cryomolds with optimal cutting temperature medium (OCT) for subsequent sectioning and histology. The degree of colon damage was graded blindly on a three-point scale corresponding to non, mild/moderate, or severe inflammation with H&E-stained sectioned colon tissue slides from healthy control, DSS treated, and DSS+VCAM1-IRAK4 NC treated mice. Histopathological assessment for inflammation grading included evaluation of alterations in the colon tissue architecture (i.e., immune cell infiltration, epithelial changes, goblet cell loss, mucosal hyperplasia, and possible crypt erosions). For immunofluorescence staining, OCT blocks of colons were cut using a CM 1860 Cryostat (Leica, Wetzlar, Germany) into 10 μm thick sections and were stained using anti-IL-6 and anti-TNF-α (504511 and 506301, BioLegend, Sandiego, CA, USA) with Goat anti-Rat IgG-Alexa594 secondary antibody (A-11007, Invitrogen, Waltham, MA, USA). NucBlue™ Fixed Cell ReadyProbes™ Reagent (ThermoFisher Scientific, Waltham, MA, USA) was used to stain the nuclei. The fluorescence images of stained sections were captured using a Keyence BZ-X710 fluorescence microscope (Keyence, Osaka, Japan). The mean fluorescence intensity was quantified with ImageJ (National Institutes of Health, Bethesda, MD). Mouse IL-6 and TNF-α ELISA kits were utilized to quantify levels of IL-6 and TNF-α proteins in colon tissue (R&D Systems, Minneapolis, MN, USA).

Statistical Analysis. The data was presented using a mean and standard deviation format (mean+/−SD), with the sample size (n) for each study specified in the figure legends. For comparisons between two groups, a two-tailed unpaired t-test was employed. For more than two groups, one-way analysis of variance (ANOVA) was used to examine the statistical significance. A statistically significant difference between groups was defined as a p-value less than 0.05 (*p<0.05), with p-values less than 0.01 (**p<0.01) and 0.001 (***p<0.001) considered highly significant. GraphPad Prism v9 (GraphPad Software, CA, USA) was used to perform all statistical analyses.

While illustrative embodiments have been illustrated and described, it will be appreciated that various changes can be made therein without departing from the spirit and scope of the invention.

The embodiments of the invention in which an exclusive property or privilege is claimed are defined as follows:

1. A nanoparticle for delivering an interleukin-1 receptor-associated kinase 4 (IRAK4) inhibitor to inflamed tissue, comprising:
(a) a first amphiphilic block polyethylene glycol-polycaprolactone (PEG-PCL) copolymer having a hydrophilic PEG block and a hydrophobic PCL block, and
(b) a second amphiphilic block polyethylene glycol-polycaprolactone (PEG-PCL) copolymer having a hydrophilic PEG block and a hydrophobic PCL block, and an IRAK4 targeting moiety covalently coupled to the terminus of the hydrophilic PEG block,
wherein the first and second copolymers form a nanoparticle having a bilayer structure with an inner hydrophobic layer (or core) formed from the first and second hydrophobic PCL blocks and an outer hydrophilic layer (or shell) formed by the first and second hydrophilic PEG blocks; and
(c) an IRAK4 inhibitor encapsulated in the hydrophobic layer (or core).

2. The nanoparticle of claim 1, wherein the ratio of the first amphiphilic block polyethylene glycol-polycaprolactone (PEG-PCL) copolymer to the second amphiphilic block polyethylene glycol-polycaprolactone (PEG-PCL) copolymer is about 9:1 (weight:weight).

3. The nanoparticle of claim 1, wherein the IRAK4 inhibitor is zimlovisertib (PF-06650833).

4. The nanoparticle of claim 1, wherein the targeting moiety is a peptide having a binding affinity to a vascular cell adhesion molecule 1 (VCAM1).

5. The nanoparticle of claim 1, wherein the targeting moiety is VHPKQHRGGSKGC (SEQ ID NO: 1).

6. A pharmaceutical composition comprising the nanoparticle of claim 1 and a pharmaceutically acceptable carrier.

7. A method for delivery of an interleukin-1 receptor-associated kinase 4 (IRAK4) inhibitor to inflamed tissue in a subject, comprising administering a therapeutically effective amount of the nanoparticle of claim 1 to a subject in need thereof.

8. The method of claim 7, wherein administering the nanoparticle comprises systemic administration.

9. The method of claim 7, wherein the subject is a human.

10. A method for treating inflamed tissue in a subject, comprising administering a therapeutically effective amount of the nanoparticle of claim 1 to a subject in need thereof.

11. The method of claim 10, wherein administering the nanoparticle comprises systemic administration.

12. The method of claim 10, wherein the subject is a human.

13. A method for treating colitis in a subject, comprising administering a therapeutically effective amount of the nanoparticle of claim 1 to a subject in need thereof.

14. The method of claim 13, wherein administering the nanoparticle comprises systemic administration.

15. The method of claim 13, wherein the subject is a human.


SEQUENCE LISTING (1 sequence)

SEQ ID NO: 1 - protein, 13 aa, Synthetic construct
VHPKQHRGGSKGC